(19) United States
(12) Patent Application Publication
(10) Pub. No.: US 2023/0233851 A1
(43) Pub. Date: Jul. 27, 2023

(54) DEVICES AND METHODS FOR TREATING CANCER BY SPLANCHNIC NERVE STIMULATION

(71) Applicant: Iota Biosciences, Inc., Alameda, CA (US)

(72) Inventors: Ryan NEELY, El Cerrito, CA (US); Michel M. MAHARBIZ, El Cerrito, CA (US); Jose M. CARMENA, Alameda, CA (US)

(21) Appl. No.: 18/007,683

(22) PCT Filed: Jun. 4, 2021
(86) PCT No.: PCT/US2021/035923

§ 371 Date: Dec. 1, 2022

Related U.S. Application Data
(60) Provisional application No. 63/034,604, filed on Jun. 4, 2020.

(51) Int. Cl. A61N 1/36; A61N 1/05; A61N 1/372; A61B 5/00

(52) U.S. Cl. CPC A61N 1/36002; A61N 1/36121; A61N 1/0556; A61N 1/37282; A61B 5/0031

(57) ABSTRACT

Methods, implantable devices, and systems for treating a cancer or inhibiting cancer growth or recurrence in a subject are described herein. Such methods can include electrically stimulating a thoracic splanchnic nerve (such as a greater splanchnic nerve) of the subject with a plurality of electrical pulses emitted from one or more electrodes m electrical communication with the splanchnic nerve, wherein the plurality of electrical pulses triggers one or more action potentials in the splanchnic nerve to increase circulating natural killer (NK) cells in the subject. An implantable device may include one or more electrodes configured to be in electrical communication with a thoracic splanchnic nerve of a subject with cancer, and be configured to operate the one or more electrodes to electrically stimulate the splanchnic nerve with a plurality of electrical pulses that triggers one or more action potentials in the splanchnic nerve that increase circulating NK cells.

### PRIORITY

This application claims priority to U.S. Patent Application Ser. No. 63/034,604 filed on Jun. 4, 2020, the disclosure of which is incorporated herein by reference in its entirety

### FIELD OF THE INVENTION

Described herein are methods and devices for treating cancer in a subject, or preventing the growth or recurrence of a cancer in the subject, by electrically stimulating a thoracic splanchnic nerve of the subject.

### BACKGROUND

Natural killer (NK) cells are elements of the innate immune system that can recognize and destroy cancer cells in the body without prior antigen presentation. Interventions that are known to increase NK activity or number, such as exercise training have been shown to be effective at reducing the occurrence and recurrence of cancer. Conversely, interventions known to depress the activity of NK cells, such as surgical stress are known to increase the risk of tumor growth and metastasis, as well as worsen patient prognosis. Surgical stress is especially problematic, as tumor resection is a common treatment for cancer. Additionally, during surgery, disruption of tumors releases cancer cells into the bloodstream which increases the risk of metastases, creating a “double jeopardy” for the patient in which they are both at risk for cancer metastasis and are experiencing a depression in the activity of natural killer cells which normally play a role in the destruction of cancer.

NK cells express several receptors that influence their activity. One such receptor is the β2-adrenoreceptor (β2-AR), which is highly enriched in the membrane of NK cells relative to other lymphocytes. β2-ARs bind with high affinity to epinephrine and, to a lesser extent, norepinephrine. Activation of β2-ARs on NK cells reduces their adhesion to endothelial cells, resulting in an increase in the number of NK cells in circulation. Additionally, activation of β2-ARs by epinephrine can also alter the activity of NK cells by increasing their cytotoxic activity against tumor cells. This pathway likely accounts for at least part of the anti-cancer benefits of physical exercise, as blockade of β2-ARs in exercising human subjects reduces both the cytotoxic activity and increase of circulating NK cells.

### BRIEF SUMMARY OF THE INVENTION

As further described herein, cancer in a subject can be treated by electrically stimulating a thoracic splanchnic nerve (e.g., a greater splanchnic nerve) of a subject with a plurality of electrical pulses emitted from one or more electrodes in electrical communication with the splanchnic nerve. The plurality of electrical pulses can trigger one or more action potentials in the splanchnic nerve, which result in an increase in the number of circulating natural killer (NK) cells in the subject, which are effective in targeting and killing cancer cells.

In an exemplary method of treating a cancer in a subject, the method comprises electrically stimulating a thoracic splanchnic nerve of the subject with a plurality of electrical pulses emitted from one or more electrodes in electrical communication with the splanchnic nerve, wherein the plurality of electrical pulses triggers one or more action potentials in the splanchnic nerve to increase circulating natural killer (NK) cells in the subject.

In an exemplary method of inhibiting cancer growth or recurrence in a subject, the method comprises electrically stimulating a thoracic splanchnic nerve of the subject with a plurality of electrical pulses emitted from one or more electrodes in electrical communication with the splanchnic nerve, wherein the plurality of electrical pulses triggers one or more action potentials in the splanchnic nerve to increase circulating natural killer (NK) cells in the subject.

In some embodiments of the above methods, the subject had previously received a cancer resection surgery.

In some embodiments, the thoracic splanchnic nerve is the greater splanchnic nerve, the lesser splanchnic nerve, or the least splanchnic nerve. In some embodiments, the thoracic splanchnic nerve is the greater splanchnic nerve.

In some embodiments of the above methods, the electrical pulses have a current of about 100 μA to about 30 mA. In some embodiments, the current is constant across the plurality of electrical pulses. In some embodiments, the electrical pulses in the plurality of electrical pulses are emitted at a frequency of about 1 Hz to about 10 kHz. In some embodiments, the plurality of electrical pulses comprises a plurality of biphasic electrical pulses. In some embodiments, the biphasic electrical pulses comprise an anodal pulse phase, a cathodal pulse phase, and an inter-phase delay. In some embodiments, the biphasic electrical pulses comprises an anodal phase followed by a cathodal phase. In some embodiments, the electrical pulses are about 5 μs to about 50 ms in length. In some embodiments, the plurality of electrical pulses comprises a plurality of pulse trains comprising two or more electrical pulses. In some embodiments, the pulse trains are separated by a quiescent period of about 100 ms to about 15 seconds. In some embodiments, the electrical pulses in the plurality of electrical pulses are tonically emitted. In some embodiments, the splanchnic nerve is electrically stimulated by the plurality of electrical pulses for a period of about 1 minute to about 60 minutes. In some embodiments, the splanchnic nerve is electrically stimulated by the plurality of electrical pulses once daily to four times daily.

In some embodiments of the above methods, the one or more electrodes are operated by an implantable device fully implanted within the subject. In some embodiments, the implantable device operates the one or more electrodes to emit the one or more electrical pulses based on a trigger signal. In some embodiments, the trigger signal is generated by the implantable device. In some embodiments, the method comprises wirelessly receiving, at the implantable device, the trigger signal. In some embodiments, the trigger signal is encoded in ultrasonic waves received by the implantable device.

In some embodiments, the trigger signal is based on one or more physiological signals detected within the subject. In some embodiments, the implantable device comprises one or more sensors configured to detect the one or more physiological signals. In some embodiments, the method comprises receiving, at the implantable device, ultrasonic waves; and emitting, from the implantable device, ultrasonic backscatter encoding information related to the one or more physiological signals. In some embodiments, the method comprises transmitting, from an external device, the ultrasonic waves received by the implantable device; receiving, at the external device, the ultrasonic backscatter encoding the information related to the one or more physiological signals; generating, at the external device, the trigger signal; transmitting, from the external device, ultrasonic waves encoding the trigger signal; and receiving, at the implantable device, the ultrasonic waves encoding the trigger signal. In some embodiments, the one or more physiological signals comprises an electrophysiological signal. In some embodiments, the electrophysiological signal comprises an electrophysiological signal transmitted by the splanchnic nerve. In some embodiments, the one or more physiological signals comprises a temperature, a pressure, a strain, a pH, or an analyte level. In some embodiments, the one or more physiological signals comprises a hemodynamic signal. In some embodiments, the hemodynamic signal comprises a diastolic blood pressure, a mean blood pressure, a systolic blood pressure, a blood oxygenation level, a heart rate, or a blood perfusion rate.

In some embodiments of any of the above methods, the method comprises converting energy from ultrasonic waves received by the implantable device into electrical energy that powers the implantable device.

In some embodiments of any of the above methods, the cancer is a metastatic cancer.

In some embodiments of any of the above methods, the method further comprises administering to the subject a NK cell activator. In some embodiments, the NK cell activator comprises IL-2, IL-6, IL-15, or IL-12, or a bioactive fragment thereof.

In some embodiments of any of the above methods, the method comprises administering to the subject a chemotherapeutic agent. In some embodiments, the subject is a human.

Further described herein is a system comprising an external device and an implantable device configured to perform the any of the above methods.

Also described herein is an implantable device comprising one or more electrodes configured to be in electrical communication with a thoracic splanchnic nerve of a subject with cancer, the device configured to operate the one or more electrodes to electrically stimulate the splanchnic nerve with a plurality of electrical pulses that triggers one or more action potentials in the splanchnic nerve that increase circulating natural killer (NK) cells in the subject. In some embodiments, the thoracic splanchnic nerve is the greater splanchnic nerve, the lesser splanchnic nerve, or the least splanchnic nerve. In some embodiments, the thoracic splanchnic nerve is the greater splanchnic nerve. In some embodiments, the device further comprises a substrate configured to at least partially wrap around the splanchnic nerve and position at least one of the one or more electrodes in electrical communication with the splanchnic nerve.

In some embodiments of the above device, the electrical pulses have a current of about 100 μA to about 30 mA. In some embodiments, the current is constant across the plurality of electrical pulses. In some embodiments, the electrical pulses in the plurality of electrical pulses are emitted at a frequency of about 1 Hz to about 10 kHz. In some embodiments, the plurality of electrical pulses comprises a plurality of biphasic electrical pulses. In some embodiments, the biphasic electrical pulses comprise an anodal pulse phase, a cathodal pulse phase, and an inter-phase delay. In some embodiments, the biphasic electrical pulses comprises an anodal phase followed by a cathodal phase. In some embodiments, the electrical pulses are about 5 μs to about 5 ms in length. In some embodiments, the plurality of electrical pulses comprises a plurality of pulse trains comprising two or more electrical pulses. In some embodiments, the pulse trains are separated by a quiescent period of about 100 ms to about 15 seconds. In some embodiments, the electrical pulses in the plurality of electrical pulses are tonically emitted.

In some embodiments of the above device, the device further comprises one or more sensors configured to detect one or more physiological signals.

In some embodiments of the above device, the device further comprises a body comprising a wireless communication system attached to the substrate. In some embodiments, the device comprises the sensor configured to detect the one or more physiological signals, and the wireless communication system is configured to wireless communicate the one or more physiological signals to a second device. In some embodiments, the body is positioned on an outer surface of the substrate. In some embodiments, the wireless communication system comprises a radiofrequency (RF) antenna. In some embodiments, the wireless communication system comprises an ultrasonic transducer. In some embodiments, the ultrasonic transducer is configured to receive ultrasonic waves and convert energy from the ultrasonic waves into electrical energy that powers the device. In some embodiments, the device comprises the one or more sensors configured to detect the one or more physiological signals, and wherein the ultrasonic transducer is configured to receive ultrasonic waves and emit ultrasonic backscatter encoding the one or more physiological signals.

In some embodiments of the above device, the device further comprises an integrated circuit configured to operate the one or more electrodes to electrically stimulate the splanchnic nerve in response to a trigger signal. In some embodiments, the device comprises the one or more sensors configured to detect the one or more physiological signals, wherein the integrated circuit is configured to generate the trigger signal using the one or more physiological signals. In some embodiments, the device comprises the wireless communication system, wherein the wireless communication system is configured to receive the trigger signal.

In some embodiments of the above device, the one or more physiological signals comprises an electrophysiological signal. In some embodiments, the electrophysiological signal comprises an electrophysiological signal transmitted by the splanchnic nerve. In some embodiments, the one or more physiological signals comprises a temperature, a pressure, a strain, a pH, or an analyte level. In some embodiments, the one or more physiological signals comprises a hemodynamic signal. In some embodiments, the hemodynamic signal comprises a diastolic blood pressure, a mean blood pressure, a systolic blood pressure, a blood oxygenation level, a heart rate, or a blood perfusion rate.

In some embodiments of the above device, the implanted device has a volume of about 5 mm³ or smaller.

Also described herein is a system that comprises any of the above devices and an interrogator comprising a wireless communication system configured to wirelessly communicate with or power the device.

### BRIEF DESCRIPTION OF THE DRAWINGS

FIG. 1 shows an exemplary board assembly for an implantable device body, which may be enclosed in a housing and attached to a substrate (such as a nerve cuff).

FIG. 2 shows a board assembly for a body of a device that includes two orthogonally positioned ultrasonic transducers.

FIG. 3 shows an exemplary body housing attached to a nerve cuff using fasteners.

FIG. 4 shows an exemplary housing with an acoustic window that may be attached to the top of the housing, and a port that may be used to fill the housing with an acoustically conductive material.

FIG. 5A shows an exemplary housing with a feedthrough port at the base of the housing. FIG. 5B shows a housing with a feedthrough attached to the housing. The feedthroughs fit through the feedthrough port, and are brazed, soldered, or otherwise attached to the housing to form a hermetic seal. FIG. 5C shows a cross-sectional view of a device with a housing attached to a nerve cuff. Feedthroughs attached to the housing electrically connect electrodes on the nerve cuff to the board assembly contained within the housing.

FIG. 6A shows an exemplary helical nerve cuff in a flexed configuration, wherein the helical portions are partially unwound. FIG. 6B shows the helical nerve cuff in FIG. 6B in a relaxed configuration, with the helical portions wound after recoiling from the flexed configuration.

FIG. 7A shows an exemplary helical nerve cuff, which may optionally be part of the implantable device described herein. FIG. 7B shows the nerve cuff illustrated in FIG. 7A from a different angle. FIG. 7C shows an exemplary helical nerve cuff similar to the nerve cuff shown in FIG. 7A and FIG. 7B, but further includes a first handle portion attached to the helical substrate proximal to a first end of the substrate, and a second handle portion attached to the helical substrate proximal to a second end of the substrate. FIG. 7D and FIG. 7E show the helical nerve cuff of FIG. 7A and FIG. 7B attached to a body having a housing. FIG. 7F shows the helical nerve cuff of FIG. 7C attached to a body having a housing.

FIG. 8A and FIG. 8B show front and back perspectives, respectively, of another embodiment of a helical nerve cuff. FIG. 8C shows the helical nerve cuff of FIG. 8A and FIG. 8B attached to a body having a housing.

FIG. 9A and FIG. 9B show front and bottom perspectives, respectively, of another embodiment of a helical nerve cuff FIG. 9C shows the helical nerve cuff of FIG. 9A and FIG. 9B attached to a body having a housing.

FIG. 10A and FIG. 10B show bottom and top perspectives, respectively, of another embodiment of a helical nerve cuff.

FIG. 11A and FIG. 11B show bottom and top perspectives, respectively, of another embodiment of a helical nerve cuff.

FIG. 12 shows an exemplary interrogator that can be used with the implantable device.

FIG. 13 shows an interrogator in communication with an implantable device. The interrogator can transmit ultrasonic waves, which can encode a trigger signal. The implantable device emits an ultrasonic backscatter, which can be modulated by the implantable device to encode information.

FIG. 14 shows plasma epinephrine concentrations before, during, and after greater splanchnic nerve stimulation. Sham stimulation indicates animals that underwent the same surgical procedures but were not stimulated. Error bars show standard error. The plot indicates that greater splanchnic nerve stimulation causes the release of epinephrine.

FIG. 15 shows a time course of the number of NK cells in the peripheral blood, measured as a percent of total lymphocytes for each greater splanchnic nerve stimulation test animal compared to sham stimulation (control) animals, which indicates that greater splanchnic nerve stimulation causes an increase in the number of circulating NK cells. Each individual point represents a blood sample from a single animal at the indicated time point.

FIG. 16 shows the fold change in the number of YAC-1 cells detected in in the lung tissue of sacrificed test (“stim”) normalized relative to the number of YAC-1 cells detected in a matched control (“sham”) animal (control animals are self-normalized).

FIG. 17 shows plasma epinephrine levels in test animals (greater splanchnic stimulation) and control animals (sham stimulation) before and after greater splanchnic nerve stimulation period, which indicates that stimulation causes the release of epinephrine.

FIG. 18 shows the number of NK cells in the peripheral blood before and after the greater splanchnic nerve stimulation period, normalized to the pre-stimulation value, which indicates that greater splanchnic nerve stimulation causes an increase in the number of circulating NK cells.

### DETAILED DESCRIPTION OF THE INVENTION

Methods for treating cancer, or for inhibiting the growth or recurrence of cancer (for example, after a cancer resection surgery), by electrically stimulating a thoracic splanchnic nerve (e.g., the greater splanchnic nerve, the lesser splanchnic nerve, or the least splanchnic nerve) a subject are described herein. The methods include increasing natural killer cell (NK) circulation in the subject by using one or more electrode in electrical communication with the splanchnic nerve, which emit a plurality of electrical pulses to electrically stimulate the splanchnic nerve.

Also described are devices and systems for performing such methods. For example, an implantable device that includes one or more electrodes configured to be in electrical communication with the splanchnic nerve of the subject is described herein. The device is configured to electrically stimulate the splanchnic nerve with a plurality of electrical pulses that increase circulating natural killer (NK) cells in the subject.

Electrical stimulation of a thoracic splanchnic nerve, such as the greater splanchnic nerve, using electrical pulses can trigger action potentials in the axons of the nerve. These action potentials then trigger the release of catecholamines (e.g., epinephrine and/or norepinephrine) from the adrenal medulla into the circulatory system, which can bind to β2-adrenoreceptors on the NK cells distributed throughout the body. Catecholamine binding results in increased NK cell mobilization. The action potentials of the stimulated greater splanchnic nerve can also activate the splenic nerve, which consequently results in the release of norepinephrine in the spleen. Because a large population of NK cells reside within the spleen, norepinephrine release in this organ results in a transient increase in the number of NK cells in circulation. Thus, greater splanchnic nerve stimulation both increases circulating NK cells by innervating the spleen, but also further activates the circulating NK cells by innervating adrenal medulla causing release of catecholamines. Once mobilized, these NK cells are then free to encounter any cancer cells that may be present in the body. After a period of minutes to hours, the NK cells then redistribute back into tissue, but preferentially attach to any cancerous tissue. For example, the mobilized NK cells can then bind to blood-borne cancer cells or can localize to solid tumors. Once the NK cells have identified cancer cells, they can begin killing the cancer cells.

### Definitions

As used herein, the singular forms “a,” “an,” and “the” include the plural reference unless the context clearly dictates otherwise.

Reference to “about” a value or parameter herein includes (and describes) variations that are directed to that value or parameter per se. For example, description referring to “about X” includes description of “X”.

A “biphasic pulse” as used herein refers to a single electrical pulse with an anodal phase and a cathodal phase, in either order, optionally with an inter-phase delay between the anodal phase and the cathodal. Reference to a length of time made with respect to the biphasic pulse refers to the length of time that includes the anodal phase, the cathodal phase, and any inter-phase delay.

The terms “individual,” “patient,” and “subject” are used synonymously, and refer to a mammal.

An “increase” or “increasing” refers to a growth in absolute numbers or a relative growth in numbers. An “increase in circulating natural killer cells” refers to an increase in the absolute number of circulating natural killer cells or an increase in the number of circulating natural killer cells relative to a total number of circulating lymphocytes.

It is understood that aspects and variations of the invention described herein include “consisting” and/or “consisting essentially of” aspects and variations.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The figures illustrate processes according to various embodiments. In the exemplary processes, some blocks are, optionally, combined, the order of some blocks is, optionally, changed, and some blocks are, optionally, omitted. In some examples, additional steps may be performed in combination with the exemplary processes. Accordingly, the operations as illustrated (and described in greater detail below) are exemplary by nature and, as such, should not be viewed as limiting.

The disclosures of all publications, patents, and patent applications referred to herein are each hereby incorporated by reference in their entireties. To the extent that any reference incorporated by reference conflicts with the instant disclosure, the instant disclosure shall control.

### Electrical Stimulation of the Thoracic Splanchnic Nerve

The thoracic splanchnic nerve can be activated to increase circulating natural killer cells in a subject by electrically stimulating the splanchnic nerve with a plurality of electrical pulses emitted from one or more electrodes in electrical communication with the splanchnic nerve. In some embodiments, the thoracic splanchnic nerve is the greater splanchnic nerve. In some embodiments, the thoracic splanchnic nerve is the lesser splanchnic nerve. In some embodiments, the thoracic splanchnic nerve is the least splanchnic nerve.

A cancer in a subject can be treated, or cancer growth or recurrence (for example, after a cancer resection surgery) can be inhibited, by increasing circulating natural killer cells in the subject by electrically stimulating the splanchnic nerve of the subject with a plurality of electrical pulses emitted from one or more electrodes in electrical communication with the splanchnic nerve. In some embodiments, the electrical stimulation triggers one or more action potentials in the splanchnic nerve.

In some embodiments, the one or more electrodes contact the splanchnic nerve. For example, the one or more electrodes may be positioned on a substrate, such as a nerve cuff, that at least partially surrounds the splanchnic nerve. Once the substrate is in position, the one or more electrodes are in electrical communication with the nerve such that, when the one or more electrodes are operated to emit electrical pulses, the electrical pulses activate the splanchnic nerve. In some embodiments, the one or more electrodes are part of a fully implantable device, for example the implantable device further described herein.

The electrical pulses may be monophasic (i.e., having only a cathodal phase or only an anodal phase) or biphasic (i.e., having both cathodal phase and anodal phase). The order of the cathodal phase and the anodal phase in a biphasic pulse may be in either order (i.e., anodal-first or cathodal-first). The anodal phase and the cathodal phase of the biphasic pulse may be separated by an interphase interval (for example about 10 μs to about 150 μs in length, such as about 10 μs to about 20 μs, about 20 μs to about 40 μs, about 40 μs to about 60 μs, about 60 μs to about 80 μs, about 80 μs to about 100 μs, or about 100 μs to about 150 μs in length). The interphase interval is generally short enough to allow for reversal of incidental redox reactions, and long enough to allow for substantial depolarization of the nerve before the charge is reversed. In some embodiments, the anodal phase and the cathodal phase of the biphasic pulse are the same length. In some embodiments, the anodal phase and the cathodal phase of the biphasic pulse are different lengths.

In some embodiments, the one or more electrical pulses comprise biphasic electrical pulses, wherein the anodal phase and cathodal phase have the same current magnitude and/or length. In some embodiments, the one or more electrical pulses comprise biphasic electrical pulses, wherein the anodal phase and cathodal phase have a different current magnitude and/or a different length. For example, in some embodiments, the one or more electrical pulses comprise biphasic electrical pulses, wherein the anodal phase has a greater current magnitude and a short length than the cathodal phase. In some embodiments, the one or more electrical pulses comprise biphasic electrical pulses, wherein the cathodal phase has a greater current magnitude and a short length than the anodal phase.

In some embodiments, the electrical pulses are about 5 μs to about 5 ms (such as about 5 μs to about 10 μs, about 10 μs to about 20 μs, about 20 μs to about 50 μs, about 50 μs to about 100 μs, about 100 μs to about 150 μs, about 150 μs to about 300 μs, about 300 μs to about 500 μs, about 500 μs to about 1 ms, or about 1 ms to about 5 ms) in length. In some embodiments, the one or more electrical pulses comprise biphasic electrical pulses, wherein the anodal phase and cathodal phase have the same length. In some embodiments, the one or more electrical pulses comprise biphasic electrical pulses, wherein the anodal phase and cathodal phase have a different a different length. In some embodiments, the anodal phase is longer than the cathodal phase. In some embodiments, the anodal phase is shorter than the cathodal phase. In some embodiments, the electrical pulses are biphasic and comprise an anodal phase about 5 μs to about 5 ms (such as about 5 μs to about 10 μs, about 10 μs to about 20 μs, about 20 μs to about 50 μs, about 50 μs to about 100 μs, about 100 μs to about 150 μs, about 150 μs to about 300 μs, about 300 μs to about 500 μs, about 500 μs to about 1 ms, or about 1 ms to about 5 ms) in length. In some embodiments, the electrical pulses are biphasic and comprise a cathodal phase about 5 μs to about 5 ms (such as about 5 μs to about 10 μs, about 10 μs to about 20 μs, about 20 μs to about 50 μs, about 50 μs to about 100 μs, about 100 μs to about 150 μs, about 150 μs to about 300 μs, about 300 μs to about 500 μs, about 500 μs to about 1 ms, or about 1 ms to about 5 ms) in length.

In some embodiments, the one or more electrical pulses have a current of about 100 microamp (μA) to about 30 mA (such as a about 100 μA to about 250 μA, about 250 μA to about 500 μA, about 500 μA to about 1 mA, about 1 mA to about 2 mA, about 2 mA to about 3 mA, about 3 mA to about 5 mA, about 5 mA to about 10 mA, about 10 mA to about 20 mA, or about 20 mA to about 30 mA). In some embodiments, the electrical pulses have the same approximately (for example, within 10%, within 5%, within 2%, or within 1% of each other) the same current across the plurality of electrical pulses.

In some embodiments, the one or more electrical pulses have a frequency of about 1 Hz to about 10 kHz (such as about 1 Hz to about 5 Hz, about 5 Hz to about 10 Hz, about 10 Hz to about 20 Hz, about 20 Hz to about 30 Hz, about 30 Hz to about 40 Hz, about 40 Hz to about 50 Hz, about 50 Hz to about 75 Hz, about 75 Hz to about 100 Hz, about 100 Hz to about 150 Hz, about 150 Hz to about 200 Hz, about 200 Hz to about 300 Hz, about 300 Hz to about 400 Hz, about 400 Hz to about 500 Hz, about 500 Hz to about 750 Hz, about 750 Hz to about 1 kHz, about 1 kHz to about 2 kHz, about 2 kHz to about 5 kHz, or about 5 kHz to about 10 kHz).

In some embodiments, the plurality of electrical pulses are tonically emitted. In some embodiments, the plurality of electrical pulses are tonically emitted at a frequency of about 1 Hz to about 10 kHz (such as about 1 Hz to about 5 Hz, about 5 Hz to about 10 Hz, about 10 Hz to about 20 Hz, about 20 Hz to about 30 Hz, about 30 Hz to about 40 Hz, about 40 Hz to about 50 Hz, about 50 Hz to about 75 Hz, about 75 Hz to about 100 Hz, about 100 Hz to about 150 Hz, about 150 Hz to about 200 Hz, about 200 Hz to about 300 Hz, about 300 Hz to about 400 Hz, about 400 Hz to about 500 Hz, about 500 Hz to about 750 Hz, about 750 Hz to about 1 kHz, about 1 kHz to about 2 kHz, about 2 kHz to about 5 kHz, or about 5 kHz to about 10 kHz).

In some embodiments, the plurality of electrical pulses are emitted in a plurality of pulse trains (i.e. in a plurality of “burst” patterns). The pulse trains include a plurality of individual electrical pulses emitted at a set frequency, and the pulse trains are separated by a quiescent period. In some embodiments, the pulse trains include 2 to about 5000 electrical pulses (for example, 2 to about 5 pulses, about 5 pulses to about 10 pulses, about 10 pulses to about 50 pulses, about 50 pulses to about 100 pulses, about 100 pulses to about 250 pulses, about 250 pulses to about 500 pulses, about 500 pulses to about 1000 pulses, about 1000 pulses to about 2500 pulses, or about 2500 pulses to about 5000 pulses.). In some embodiments the pulse trains include about 5 to about 5000 electrical pulse. In some embodiments, the pulse trains include about 5 to about 500 electrical pulses. For example, the pulse trains may be separated by a quiescent period of about 100 ms to about 15 seconds (such as about 100 ms to about 250 ms, about 250 ms to about 500 ms, about 500 ms to about 1 second, about 1 second to about 2 seconds, about 2 seconds to about 5 seconds, about 5 seconds to about 10 seconds, or about 10 seconds to about 15 seconds). In some embodiments, the electrical pulses within the pulse train are emitted at a frequency of about 1 Hz to about 10 kHz (such as about 1 Hz to about 5 Hz, about 5 Hz to about 10 Hz, about 10 Hz to about 20 Hz, about 20 Hz to about 30 Hz, about 30 Hz to about 40 Hz, about 40 Hz to about 50 Hz, about 50 Hz to about 75 Hz, about 75 Hz to about 100 Hz, about 100 Hz to about 150 Hz, about 150 Hz to about 200 Hz, about 200 Hz to about 300 Hz, about 300 Hz to about 400 Hz, about 400 Hz to about 500 Hz, about 500 Hz to about 750 Hz, about 750 Hz to about 1 kHz, about 1 kHz to about 2 kHz, about 2 kHz to about 5 kHz, or about 5 kHz to about 10 kHz).

The electrical pulses may be episodically emitted from the one or more electrodes to stimulate the splanchnic nerve. Episodic stimulation allows an increase in natural killer cell circulation, which, once in circulation, can localize to cancerous tissue, which may occur over the course of several minutes to several hours. In some embodiments, the splanchnic nerve is electrically stimulated by the plurality of electrical pulses for a period of about 1 minute to about 60 minutes (such as about 1 minute to about 2 minutes, about 2 minutes to about 5 minutes, about 5 minutes to about 10 minutes, about 10 minutes to about 15 minutes, about 15 minutes to about 20 minutes, about 20 minutes to about 30 minutes, about 30 minutes to about 45 minutes, or about 45 minutes to about 60 minutes. In some embodiments, episodic stimulation occurs at a frequency from once daily to once hourly, or a frequency in between (for example, once every two hours, once every three hours, once every four hours, once every six hours, once every eight hours, or once every 12 hours). In some embodiments, episodic stimulation occurs at a frequency from once daily to four times daily.

The electrical pulses administered to the splanchnic nerve may be sinusoidal, square, sawtooth, or any other suitable shape.

In some embodiments, a method of treating a cancer in a subject includes increasing circulating natural killer (NK) cells in the subject by electrically stimulating a thoracic splanchnic nerve of the subject with a plurality of biphasic electrical pulses emitted from one or more electrodes in electrical communication with the splanchnic nerve. In some embodiments, the thoracic splanchnic nerve is the greater splanchnic nerve. In some embodiments, the one or more electrodes are operated by an implantable device fully implanted within the subject. In some embodiments, the subject had previously received a cancer resection surgery.

In some embodiments, a method of treating a cancer in a subject includes increasing circulating natural killer (NK) cells in the subject by electrically stimulating a thoracic splanchnic nerve of the subject with a plurality of biphasic electrical pulses emitted at a frequency of about 1 Hz to about 10 kHz from one or more electrodes in electrical communication with the splanchnic nerve. In some embodiments, the thoracic splanchnic nerve is the greater splanchnic nerve. In some embodiments, the one or more electrodes are operated by an implantable device fully implanted within the subject. In some embodiments, the subject had previously received a cancer resection surgery.

In some embodiments, a method of treating a cancer in a subject includes increasing circulating natural killer (NK) cells in the subject by electrically stimulating a thoracic splanchnic nerve of the subject with a plurality of biphasic electrical pulses tonically emitted at a frequency of about 1 Hz to about 10 kHz from one or more electrodes in electrical communication with the splanchnic nerve. In some embodiments, the thoracic splanchnic nerve is the greater splanchnic nerve. In some embodiments, the one or more electrodes are operated by an implantable device fully implanted within the subject. In some embodiments, the subject had previously received a cancer resection surgery.

In some embodiments, a method of treating a cancer in a subject includes increasing circulating natural killer (NK) cells in the subject by electrically stimulating a thoracic splanchnic nerve of the subject with a plurality of biphasic electrical pulses tonically emitted at a frequency of about 1 Hz to about 10 kHz from one or more electrodes in electrical communication with the splanchnic nerve, wherein the electrical pulses are about 5 μs to about 5 ms in length. In some embodiments, the thoracic splanchnic nerve is the greater splanchnic nerve. In some embodiments, the one or more electrodes are operated by an implantable device fully implanted within the subject. In some embodiments, the subject had previously received a cancer resection surgery.

In some embodiments, a method of treating a cancer in a subject includes increasing circulating natural killer (NK) cells in the subject by electrically stimulating a thoracic splanchnic nerve of the subject with a plurality of biphasic electrical pulses tonically emitted at a frequency of about 1 Hz to about 10 kHz from one or more electrodes in electrical communication with the splanchnic nerve, wherein the electrical pulses are about 5 μs to about 5 ms in length. In some embodiments, the thoracic splanchnic nerve is the greater splanchnic nerve. In some embodiments, the one or more electrodes are operated by an implantable device fully implanted within the subject. In some embodiments, the subject had previously received a cancer resection surgery.

In some embodiments, a method of treating a cancer in a subject includes increasing circulating natural killer (NK) cells in the subject by electrically stimulating a thoracic splanchnic nerve of the subject with a plurality of biphasic electrical pulses tonically emitted at a frequency of about 1 Hz to about 10 kHz from one or more electrodes in electrical communication with the splanchnic nerve, wherein the electrical pulses are about 5 μs to about 5 ms in length and have an constant current of about 100 μA to about 30 mA. In some embodiments, the thoracic splanchnic nerve is the greater splanchnic nerve. In some embodiments, the one or more electrodes are operated by an implantable device fully implanted within the subject. In some embodiments, the subject had previously received a cancer resection surgery.

In some embodiments, a method of inhibiting cancer growth or recurrence in a subject includes increasing circulating natural killer (NK) cells in the subject by electrically stimulating a thoracic splanchnic nerve of the subject with a plurality of biphasic electrical pulses emitted from one or more electrodes in electrical communication with the splanchnic nerve. In some embodiments, the thoracic splanchnic nerve is the greater splanchnic nerve. In some embodiments, the one or more electrodes are operated by an implantable device fully implanted within the subject. In some embodiments, the subject had previously received a cancer resection surgery.

In some embodiments, a method of inhibiting cancer growth or recurrence in a subject includes increasing circulating natural killer (NK) cells in the subject by electrically stimulating a thoracic splanchnic nerve of the subject with a plurality of biphasic electrical pulses emitted at a frequency of about 1 Hz to about 10 kHz from one or more electrodes in electrical communication with the splanchnic nerve. In some embodiments, the thoracic splanchnic nerve is the greater splanchnic nerve. In some embodiments, the one or more electrodes are operated by an implantable device fully implanted within the subject. In some embodiments, the subject had previously received a cancer resection surgery.

In some embodiments, a method of inhibiting cancer growth or recurrence in a subject includes increasing circulating natural killer (NK) cells in the subject by electrically stimulating a thoracic splanchnic nerve of the subject with a plurality of biphasic electrical pulses tonically emitted at a frequency of about 1 Hz to about 10 kHz from one or more electrodes in electrical communication with the splanchnic nerve. In some embodiments, the thoracic splanchnic nerve is the greater splanchnic nerve. In some embodiments, the one or more electrodes are operated by an implantable device fully implanted within the subject. In some embodiments, the subject had previously received a cancer resection surgery.

In some embodiments, a method of inhibiting cancer growth or recurrence in a subject includes increasing circulating natural killer (NK) cells in the subject by electrically stimulating a thoracic splanchnic nerve of the subject with a plurality of biphasic electrical pulses tonically emitted at a frequency of about 1 Hz to about 10 kHz from one or more electrodes in electrical communication with the splanchnic nerve, wherein the electrical pulses are about 5 μs to about 50 ms in length. In some embodiments, the thoracic splanchnic nerve is the greater splanchnic nerve. In some embodiments, the one or more electrodes are operated by an implantable device fully implanted within the subject. In some embodiments, the subject had previously received a cancer resection surgery.

In some embodiments, a method of inhibiting cancer growth or recurrence in a subject includes increasing circulating natural killer (NK) cells in the subject by electrically stimulating a thoracic splanchnic nerve of the subject with a plurality of biphasic electrical pulses tonically emitted at a frequency of about 1 Hz to about 10 kHz from one or more electrodes in electrical communication with the splanchnic nerve, wherein the electrical pulses are about 5 μs to about 50 ms in length. In some embodiments, the thoracic splanchnic nerve is the greater splanchnic nerve. In some embodiments, the one or more electrodes are operated by an implantable device fully implanted within the subject. In some embodiments, the subject had previously received a cancer resection surgery.

In some embodiments, a method of inhibiting cancer growth or recurrence in a subject includes increasing circulating natural killer (NK) cells in the subject by electrically stimulating a thoracic splanchnic nerve of the subject with a plurality of biphasic electrical pulses tonically emitted at a frequency of about 1 Hz to about 10 kHz from one or more electrodes in electrical communication with the splanchnic nerve, wherein the electrical pulses are about 5 μs to about 50 ms in length and have an constant current of about 100 μA to about 30 mA. In some embodiments, the thoracic splanchnic nerve is the greater splanchnic nerve. In some embodiments, the one or more electrodes are operated by an implantable device fully implanted within the subject. In some embodiments, the subject had previously received a cancer resection surgery.

Electrical stimulation of a thoracic splanchnic nerve can occur in response to a trigger signal received by or generated by the implantable device. The trigger signal may include instructions that include a frequency, amplitude, length, pulse pattern, and/or pulse shape of the electrical pulses emitted by the implantable device. In some embodiments, the trigger signal is wirelessly received by the implantable device, which can be transmitted by a second device (which, in some embodiments, is external to the subject). For example, the trigger signal may be communicated to the implantable device may be encoded in radiofrequency (RF), ultrasonic waves, or other wireless telemetry method. In some embodiments, the trigger signal may be generated by the implantable device itself, for example using information (e.g., one or more physiological signals) detected by or communicated to the implantable device.

The trigger signal can be based activity of a thoracic splanchnic nerve (e.g., a greater splanchnic nerve), a change in an immune system status, an increase or decrease in inflammation, an inflammatory response, or one or more physiological signals detected within the subject. In some embodiments, the trigger signal is based on one or more physiological signals. Exemplary physiological signals include an electrophysiological signal (for example, an electrophysiological signal transmitted by the splanchnic nerve, the splenic nerve, or other nerve), a temperature, a pressure, a strain, a pH, an analyte lave (for example, the presence or concentration of the analyte), or a hemodynamic signal (e.g., a diastolic blood pressure, a mean blood pressure, a systolic blood pressure, a blood oxygenation level, a heart rate, or a blood perfusion rate).

As further described herein, the implantable device can be configured to detect a physiological signal, and wirelessly transmit a signal (for example, by ultrasonic backscatter) that encodes information related to the physiological signal. The signal encoding the physiological signal can be received by a second device (for example, an interrogator as further described herein), which can decode the signal to obtain the information related to the detected physiological signal. The information can be analyzed by the second device or relayed to another computer system to analyze the information. Based on the detected physiological signal, the second device can transmit the trigger signal to the implanted device, instructing the implantable device to electrically stimulate the splanchnic nerve.

In some embodiments, the trigger signal is based on a change in splanchnic nerve activity compared to a baseline splanchnic nerve activity. A baseline splanchnic nerve activity can be established in an individual subject, for example, and the trigger signal can be based on deviations from the baseline splanchnic nerve activity. The trigger signal can be based on, for example, a voltage potential change or a voltage potential change pattern measured from the splanchnic nerve over a period of time. The voltage change (e.g., a voltage spike) is indicative of the action potential passing through the splanchnic nerve, which is detected by the electrodes on the implanted device. A difference in the frequency and/or amplitude of the voltage spike (a single voltage spike or a compound voltage spike of the action potential) can indicate a change in immune activity.

In some embodiments, the trigger signal is based on an analysis of splanchnic nerve activity patterns and a detected physiological signal, such as temperature, pulse, or blood pressure. The splanchnic nerve activity may be detected by the implantable device or by some other device or method.

In some embodiments, the trigger signal can be based on information related to aggregate information (e.g., splanchnic nerve activity and/or physiological signal) detected over a trailing period of time, for example over a period of minutes, hours, or days. For example, in some embodiments, the trigger is based on information related to splanchnic nerve activity detected from within about 30 seconds, about 1 minute, about 5 minutes about 15 minutes, about 30 minutes, about 1 hour, about 2 hours, about 4 hours, about 8 hours, about 12 hours, about 24 hours, about 2 days, about 4 days, or about 7 days.

In some embodiments, the implanted device can be operated using an interrogator, which can transmit ultrasonic waves that power and operate the implanted device. As further described herein, the interrogator is a device that includes an ultrasonic transducer that can transmit ultrasonic waves to the implanted device and/or receive ultrasonic backscatter emitted from the implanted device. In some embodiments, the interrogator is a device external to the subject, and can be worn by the subject. In some embodiments, the ultrasonic waves transmitted by the interrogator encode the trigger signal.

In some embodiments, the methods described herein are used to treat cancer in a subject, or to inhibit cancer growth or recurrence in the subject. For example, the subject may have undergone a cancer resection surgery. Non-resectable or metastatic cancer may remain within the subject, even after surgery, and there remains some risk that this residual cancer can recur and/or grow without therapeutic intervention. Further, the surgery may lower the subject immune response, making the subject even more susceptible to cancer recurrence or growth. Using the methods described herein, a thoracic (e.g., greater) splanchnic nerve can be electrically stimulated to increase circulating natural killer cells in the subject, which can target the residual cancer.

The subject receiving cancer is generally a mammal, such as a human, rat, mouse, dog, cat, horse, pig, etc.

NK cells are components of the innate immune system and can identify and destroy cells with oncologic mutations upon first contact and without prior priming or exposure. This is a unique feature of NK cells; other tumor-killing lymphocytes, such as T-cells, require prior antigen exposure before becoming cytotoxic against cancerous cells. A key property of NK cell recognition of tumor cells is the lack or downregulation of MHC Class I molecules which occurs during oncologic mutation (“missing-self” recognition). However, cancerous cells can also overexpress many other ligands which can activate NK cells. These properties allow NK cells to destroy a broad range of spontaneous, transplantable, haematopoietic and non-haematopoietic tumor cells. See, for example, Guilerey et al., Targeting natural killer cells in cancer immunotherapy, Nat. Immunol., vol. 17, pp 1025-1036 (2016); Smyth et al., New aspects of natural-killer-cell surveillance and therapy of cancer, Nat. Rev. Cancer., vol. 2, pp. 850-861 (2002); and Vesely et al., Natural innate and adaptive immunity to cancer, Annu. Rev. Immuol., vol. 29, pp. 235-271 (2011). In some embodiments, the cancer is a primary cancer. In some embodiments, the cancer is a metastatic cancer. In some embodiments, the cancer is a solid cancer. In some embodiments, the cancer is a lymphoma. Exemplary cancers include, but are not limited to, adenocortical carcinoma, agnogenic myeloid metaplasia, AIDS-related cancers (e.g., AIDS-related lymphoma), anal cancer, appendix cancer, astrocytoma (e.g., cerebellar and cerebral), basal cell carcinoma, bile duct cancer (e.g., extrahepatic), bladder cancer, bone cancer, (osteosarcoma and malignant fibrous histiocytoma), brain tumor (e.g., glioma, brain stem glioma, cerebellar or cerebral astrocytoma (e.g., pilocytic astrocytoma, diffuse astrocytoma, anaplastic (malignant) astrocytoma), malignant glioma, ependymoma, oligodenglioma, meningioma, craniopharyngioma, haemangioblastomas, medulloblastoma, supratentorial primitive neuroectodermal tumors, visual pathway and hypothalamic glioma, and glioblastoma), breast cancer, bronchial adenomas/carcinoids, carcinoid tumor (e.g., gastrointestinal carcinoid tumor), carcinoma of unknown primary, central nervous system lymphoma, cervical cancer, colon cancer, colorectal cancer, chronic myeloproliferative disorders, endometrial cancer (e.g., uterine cancer), ependymoma, esophageal cancer, Ewing's family of tumors, eye cancer (e.g., intraocular melanoma and retinoblastoma), gallbladder cancer, gastric (stomach) cancer, gastrointestinal carcinoid tumor, gastrointestinal stromal tumor (GIST), germ cell tumor, (e.g., extracranial, extragonadal, ovarian), gestational trophoblastic tumor, head and neck cancer, hepatocellular (liver) cancer (e.g., hepatic carcinoma and heptoma), hypopharyngeal cancer, islet cell carcinoma (endocrine pancreas), laryngeal cancer, laryngeal cancer, leukemia, lip and oral cavity cancer, oral cancer, liver cancer, lung cancer (e.g., small cell lung cancer, non-small cell lung cancer, adenocarcinoma of the lung, and squamous carcinoma of the lung), lymphoid neoplasm (e.g., lymphoma), medulloblastoma, ovarian cancer, mesothelioma, metastatic squamous neck cancer, mouth cancer, multiple endocrine neoplasia syndrome, myelodysplastic syndromes, myelodysplastic/myeloproliferative diseases, nasal cavity and paranasal sinus cancer, nasopharyngeal cancer, neuroblastoma, neuroendocrine cancer, oropharyngeal cancer, ovarian cancer (e.g., ovarian epithelial cancer, ovarian germ cell tumor, ovarian low malignant potential tumor), pancreatic cancer, parathyroid cancer, penile cancer, cancer of the peritoneal, pharyngeal cancer, pheochromocytoma, pineoblastoma and supratentorial primitive neuroectodermal tumors, pituitary tumor, pleuropulmonary blastoma, lymphoma, primary central nervous system lymphoma (microglioma), pulmonary lymphangiomyomatosis, rectal cancer, renal cancer, renal pelvis and ureter cancer (transitional cell cancer), rhabdomyosarcoma, salivary gland cancer, skin cancer (e.g., non-melanoma (e.g., squamous cell carcinoma), melanoma, and Merkel cell carcinoma), small intestine cancer, squamous cell cancer, testicular cancer, throat cancer, thymoma and thymic carcinoma, thyroid cancer, tuberous sclerosis, urethral cancer, vaginal cancer, vulvar cancer, Wilms' tumor, and post-transplant lymphoproliferative disorder (PTLD), abnormal vascular proliferation associated with phakomatoses, edema (such as that associated with brain tumors), and Meigs' syndrome. In some embodiments, the cancer is a leukemia.

In some embodiments, one or more natural killer cell activators are administered to the subject. The natural killer cell activator can increase the proportion of circulating NK cells in the subject, which can increase the cytotoxic effect of the NK cells towards the cancer. Exemplary NK cell activators include interleukin-2 (IL-2), interleukin-6 (IL-6), interleukin-15 (IL-15), and interleukin-12 (IL-12), or bioactive fragments, variants, or fusions thereof. A “bioactive fragment” of a NK cell activator refers to any fragment of the NK cell activator that can activate circulating NK cells. In some embodiments, the IL-2 is aldesleukin, teceleukin, bioleukin, or denileukin diftitox.

In some embodiments, there is a pharmaceutical composition comprising a natural killer (NK) cell activator for use in a method of treating a cancer in a subject, wherein the method comprises administering to the subject the pharmaceutical composition comprising the NK cell activator; and electrically stimulating a thoracic splanchnic nerve of the subject with a plurality of electrical pulses emitted from one or more electrodes in electrical communication with the splanchnic nerve, wherein the plurality of electrical pulses triggers one or more action potentials in the greater splanchnic nerve to increase circulating natural killer (NK) cells in the subject. In some embodiments, the thoracic splanchnic nerve is the greater splanchnic nerve. In some embodiments, the NK cell activator is interleukin-2 (IL-2), interleukin-6 (IL-6) interleukin-15 (IL-15), and interleukin-12 (IL-12), or a bioactive fragment thereof.

In some embodiments, there is a pharmaceutical composition comprising a natural killer (NK) cell activator for use in a method of inhibiting cancer growth or recurrence in a subject, wherein the method comprises administering to the subject the pharmaceutical composition comprising the NK cell activator; and electrically stimulating a thoracic splanchnic nerve of the subject with a plurality of electrical pulses emitted from one or more electrodes in electrical communication with the splanchnic nerve, wherein the plurality of electrical pulses triggers one or more action potentials in the greater splanchnic nerve to increase circulating natural killer (NK) cells in the subject. In some embodiments, the thoracic splanchnic nerve is the greater splanchnic nerve. In some embodiments, the NK cell activator is interleukin-2 (IL-2), interleukin-6 (IL-6) interleukin-15 (IL-15), and interleukin-12 (IL-12), or a bioactive fragment thereof.

In some embodiments, one or more chemotherapeutic agents are further administered to the subject. Exemplary chemotherapeutic agents include nucleoside analogs (such as azacitidine, capecitabine, carmofur, cladribine, clofarabine, cytarabine, decitabine, floxuridine, fludarabine, fluorouracil, gemcitabine, mercaptopurine, nelarabine, pentostati, tegafur, and tioguanine), antifolates (such as methotrexate, pemetrexed, raltitrexed), hydroxycarbamide, topoisomerase I inhibitors (such as irinotecan and topotecan), anthracyclines (such as daunorubicin, doxorubicin, epirubicin, idarubicin, mitoxantrone, and valrubicin), podophyllotoxins (such as etoposide and teniposide), taxanes (such as cabazitaxel, docetaxel, and paclitaxel), vinca alkaloids (such as vinblastine, vincristine, vindesine, vinflunine, and vinorelbine), alkylating agents (such as bendaustine, busulfan, carmustine, chlorambucil, chlormethine, cyclophosphamide, dacarbazine, fotemustine, ifosfamide, lomustine, melphalan, streptozotocin, and temozolomide), plantinum-containing agents (such as carboplatin, cisplatin, nedaplatin, oxaliplatin), altretamine, bleomycin, bortezomib, dactinomycin, estramustine, ixabepilone, mitomycin, and procarbazine), monoclonal antibodies (such as anti-CD52 antibodies (e.g., alemtuzumab), anti-VEGF antibodies (e.g., bevacizumab), anti-EGFR antibodies (e.g., cetuximab and panitumumab), anti-RANKL antibodies (such as denosumab), anti-CD33 antibodies (such as gemtuzumab ozogamicin), anti-CD20 antibodies (such as ibritumomab tiuxetan, ofatumumab, rituximab, ad tositumomab), anti-CTLA4 antibodies (such as ipilimumab), anti-PDL-1 antibodies (such as pembrolizumab), anti-HER2 inhibitors (such as pertuzumab and trastuzumab)), tyrosine kinase inhibitors (such as afatinib, afibercept, axitinib, bosutinib, crizotinib, dasatinib, erlotinib, gefitinib, imatinib, lapatinib, nilotinib, pazopanib, ponatinib, regorafenib, ruxolitinib, sorafenib, sunitinib, and vandetanib), mTOR inibitors (such as everolimus and temsirolimus), retinoids (such as alitretinoin, bexarotene, isotretinoin, tamibarotene, and tretinoin), immunomodulatory agents (such as lenalidomide, pomalidomide, and thalidomide), histone deacetylase inhibitors (such as panobinostat, romidepsin, valproate, and vorinostat), and vemurafenib.

In some embodiments, there is a pharmaceutical composition comprising a chemotherapeutic agent for use in a method of treating a cancer in a subject, wherein the method comprises administering to the subject the composition comprising the chemotherapeutic agent; and electrically stimulating a thoracic splanchnic nerve of the subject with a plurality of electrical pulses emitted from one or more electrodes in electrical communication with the greater splanchnic nerve, wherein the plurality of electrical pulses triggers one or more action potentials in the splanchnic nerve to increase circulating natural killer (NK) cells in the subject. In some embodiments, the thoracic splanchnic nerve is the greater splanchnic nerve. In some embodiments, the NK cell activator is interleukin-2 (IL-2), interleukin-6 (IL-6) interleukin-15 (IL-15), and interleukin-12 (IL-12), or a bioactive fragment thereof.

In some embodiments, there is a pharmaceutical composition comprising a chemotherapeutic agent for use in a method of inhibiting cancer growth or recurrence in a subject, wherein the method comprises administering to the subject the pharmaceutical composition comprising the chemotherapeutic agent; and electrically stimulating a thoracic splanchnic nerve of the subject with a plurality of electrical pulses emitted from one or more electrodes in electrical communication with the splanchnic nerve, wherein the plurality of electrical pulses triggers one or more action potentials in the greater splanchnic nerve to increase circulating natural killer (NK) cells in the subject. In some embodiments, the thoracic splanchnic nerve is the greater splanchnic nerve. In some embodiments, the NK cell activator is interleukin-2 (IL-2), interleukin-6 (IL-6) interleukin-15 (IL-15), and interleukin-12 (IL-12), or a bioactive fragment thereof.

### Implantable Devices and Systems

The implanted device includes one or more electrodes that are configured to be in electrical communication with the thoracic splanchnic nerve. The device is configured to operate the one or more electrodes to electrically stimulate the splanchnic nerve with a plurality of electrical pulses that increase circulating natural killer (NK) cells in the subject. In some embodiments, the implantable device is configured to perform any one or more of the methods described herein. In some embodiments, the thoracic splanchnic nerve is the greater splanchnic nerve. In some embodiments, the thoracic splanchnic nerve is the lesser splanchnic nerve. In some embodiments, the thoracic splanchnic nerve is the least splanchnic nerve.

The implantable device can include a substrate (such as a nerve cuff, which may be, for example, a helical nerve cuff) configured to position the one or more of the electrodes in electrical communication with the thoracic splanchnic nerve. For example, the substrate can include one or more of the electrodes, and be configured to at least partially wrap around the thoracic splanchnic nerve. In some embodiments, the substrate is configured to position the one or more electrodes in electrical communication with the greater splanchnic nerve. In some embodiments, the substrate is configured to position the one or more electrodes in electrical communication with the lesser splanchnic nerve. In some embodiments, the substrate is configured to position the one or more electrodes in electrical communication with the least splanchnic nerve.

In some embodiments, the implanted device includes a body, which can contain a wireless communication system (e.g., one or more ultrasonic transducers or one or more radiofrequency antennas) and/or an integrated circuit that operates the device. The wireless communication system can transmit information, such as information related to a detected physiological signal, a status of the device, and/or electrical pulses emitted from the one or more electrodes. Exemplary physiological signals that may be detected by the device and/or communicated by the wireless communication include an electrophysiological signal (for example, an electrophysiological signal transmitted by the greater splanchnic nerve, the splenic nerve, or other nerve), a temperature, a pressure, a strain, a pH, an analyte lave (for example, the presence or concentration of the analyte), or a hemodynamic signal (e.g., a diastolic blood pressure, a mean blood pressure, a systolic blood pressure, a blood oxygenation level, a heart rate, or a blood perfusion rate).

In some embodiments, the implantable device includes an ultrasonic transducer configured to receive ultrasonic waves, and convert the received ultrasonic waves into an electrical energy that powers the device. The body of the device can include or be connected to one or more electrodes and/or a sensor configured to detect a physiological signal, which are in electric communication with the ultrasonic transducer (e.g., through the integrated circuit). In some embodiments, an electric current that flows through the transducer can be modulated to encode information in ultrasonic backscatter waves emitted by the wireless communication system. The encoded information may include, for example, data related to a physiological signal detected by the sensor, a status of the device (for example, a status confirming the device is receiving signals encoded in ultrasonic waves, confirming operation of the integrated circuit, or confirming that the device is being powered), or information related to an electrical pulse emitted by the implantable device.

In some embodiments, the implantable device comprises a substrate (such as a nerve cuff) attached to the body that is sized and configured to attach the device to the thoracic splanchnic nerve. In some embodiments, the thoracic splanchnic nerve is the greater splanchnic nerve, the lesser splanchnic nerve, or the leas splanchnic nerve. The body may be attached to the nerve cuff, for example, by positioning the body (which may include a housing) on the outer surface of the nerve cuff. The nerve cuff is further sized and configured to position electrodes in electrical communication with the splanchnic nerve. In some embodiments, the nerve cuff is configured to at least partially surround the splanchnic nerve and position the two or more electrodes in electrical communication with the splanchnic nerve.

The implantable device may be part of system that further includes a second device, such as an interrogator as further described herein. The second device may be an external device. In some embodiments, the second device of the system sends a trigger signal to the implantable device providing instructions to the implantable device for emitting the plurality of electrical pulses from the one or more electrodes. In some embodiments, the implantable device can wirelessly transmit information related to a physiological signal detected by the implantable device to the second device, for example using radiofrequency or ultrasonic backscatter. The second device, in some embodiments, is configured to receive the information related to the detected physiological signal, and generate the trigger signal based on the related information. Thus, the system can, in some embodiments, form a closed-loop system that electrically stimulates the splanchnic nerve based on a physiological signal detected by the implantable device.

In some embodiments, the implantable device itself can generate the trigger signal providing instructions to operate the one or more electrodes to emit a plurality of electrical pulses that electrically stimulated the splanchnic nerve to increase circulating natural killer cells. The implantable device may be full implantable, and can be part of a system further including a second device (which may be an external device) that can power the implantable device. For example, the second device may be an interrogator that includes one or more ultrasonic transducers that can emit ultrasonic waves, which can be received by an ultrasonic transducer of the implantable device and converted into an electrical energy that powers the implantable device.

In some embodiments, the one or more electrodes comprise one or more monopolar electrodes. In some embodiments, the one or more electrodes comprise one or more bipolar electrodes. In some embodiments, the one or more electrodes comprise tripolar electrodes. The biopolar or tripolar electrodes may be particularly beneficial, for example, to contain the stimulation current and prevent it from activating nearby musculature.

In some embodiments, the implantable device is configured to be anchored to adjacent tissue. For example, the implantable device may include a substrate that positions the one or more electrodes in electrical communication with the splanchnic nerve, and may further be anchored to adjacent tissue. Anchoring the device to adjacent tissue helps keep the device in the implanted position.

### Body of the Implantable Device

The implantable device can include a body attached to a substrate (such as a nerve cuff) configured to engage the thoracic splanchnic nerve. The body may be attached to the substrate without an interceding lead between the body and the substrate. That is, the body can be positioned on the outer surface of the nerve cuff such that the body and the nerve cuff are simultaneously positioned when implanted in the body. The body may include a wireless communication system, which is electrically connected to the one or more electrodes that are configured to emit a plurality of electrical pulses to electrically stimulate the splanchnic nerve. In some embodiments, one of the one or more electrodes is positioned on the body of the implantable device, and one of the one or more electrodes is positioned on the substrate. The substrate may be, for example, a helical nerve cuff as described in further detail herein. The implantable device is fully implantable; that is, no wires or leads connect outside the body of the subject after implantation.

The one or more electrodes configured to electrically stimulate the splanchnic nerve, including one or more of the electrodes on the substrate, are electrically connected to the wireless communication system. The body of the device may further include an integrated circuit, and the electrodes are connected to the wireless communication system through integrated circuit. The integrated circuit may be configured to operate the wireless communication system of the device body, and can operate the one or more electrodes of the implantable device to emit the plurality of electrical pulses. Optionally, the implantable device includes one or more sensors configured to detect a physiological signal (such as a temperature sensor, an oxygen sensor, a pH sensor, a strain sensor, a pressure sensor, an impedance sensor, or a sensor that can detect a concentration of an analyte). In some embodiments, the sensor configured to detect a physiological signal includes one or more electrodes configured to detect an electrophysiological signal, such as an electrophysiological signal transmitted by the splanchnic nerve. In some embodiments, one or more of the electrodes are positioned on the body of the implantable device. In some embodiments, one or more of the electrodes are positioned on the substrate of the device.

The body of the implantable device may include a wireless communication system, which can communicate with a separate device (such as an external interrogator or another implantable device). For example, the wireless communication may be configured to receive instructions for emitting the electrical pulses to the splanchnic nerve (i.e., a trigger signal) and/or transmit information, such as data associated with detected physiological signal. The wireless communication system can include, for example one or more ultrasonic transducers or one or more radiofrequency antennas. The wireless communication system may also be configured to receive energy (for example, through ultrasonic waves or radiofrequency (RF)) from another device, which can, in some embodiments, also be used to power the implantable device.

Information about the detected physiological signal may be transmitted using the wireless communication system to a receiving device. For example, the wireless communication system may include an ultrasonic transducer, which can be operated to encode information about the detected physiological signal using ultrasonic backscatter waves or radiofrequency backscatter waves. Exemplary implantable devices that can detect an electrophysiological signal and encode information related to the detected electrophysiological signal are described in WO 2018/009910 A2. Exemplary implantable devices that can be operated using ultrasonic waves to emit an electrical pulse are described in WO 2018/009912 A2. Exemplary implantable devices that are powered by ultrasonic waves and can emit an ultrasonic backscatter encoding a detected physiological signal are described in WO 2018/009905 A2 and WO 2018/009911 A2.

An integrated circuit included in the device body can electrically connect and communicate between the electrodes or sensor and the wireless communication system (e.g., the one or more ultrasonic transducers or one or more RF antennas). The integrated circuit can include or operate a modulation circuit within the wireless communication system, which modulates an electrical current flowing through the wireless communication system (e.g., one or more ultrasonic transducers or one or more radiofrequency antennas) to encode information in the electrical current. The modulated electrical current affects backscatter waves (e.g., ultrasonic backscatter waves or radiofrequency backscatter waves) emitted by the wireless communication system, and the backscatter waves encode the information.

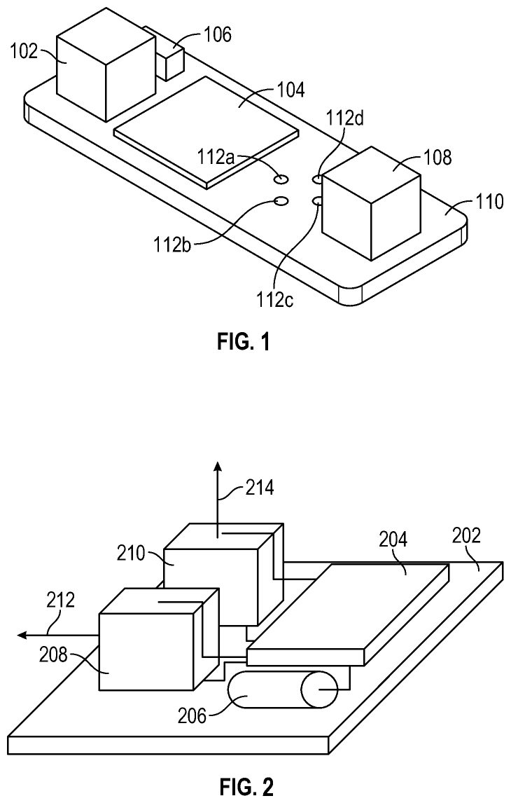

FIG. 1 shows a side view of an exemplary board assembly for an implantable device body, which may be surrounded by a housing, and can be attached to a nerve cuff. The board assembly includes a wireless communication system (e.g., an ultrasonic transducer) 102 and an integrated circuit 104. In the illustrated embodiment, the integrated circuit 104 includes a power circuit that includes a capacitor 106. In the illustrated embodiment, the capacitor is an “off chip” capacitor (in that it is not on the integrated circuit chip), but is still electrically integrated into the circuit. The capacitor can temporarily store electrical energy converted from energy (e.g., ultrasonic waves) received by the wireless communication system, and can be operated by the integrated circuit 104 to store or release energy. Optionally, the body further includes a sensor 108, configured to detect a physiological signal. The ultrasonic transducer 102, integrated circuit 104, the capacitor 106, and the optional sensor 108 are mounted on a circuit board 110, which may be a printed circuit board. The circuit board 110 may further include one or more feedthroughs 112a, 112b, 112c, and 112d that electrically connect the circuit board and/or integrated circuit to one or more electrodes of the nerve cuff. The wireless communication system 102 is electrically connected to the integrated circuit 104, and the integrated circuit 104 is electrically connected to the electrodes via the feedthroughs 112a, 112b, 112c, and 112d, thereby electrically connecting the wireless communication system 102 to the electrodes.

The wireless communication system can be configured to receive instructions for operating the implantable device. The instructions may be transmitted, for example, by a separate device, such as an interrogator. By way of example, ultrasonic waves received by the implantable device (for example, those transmitted by the interrogator) can encode instructions for operating the implantable device. In another example, RF waves received by the implantable device can encode instructions for operating the implantable device. The instructions may include, for example, a trigger signal that instructs the implantable device to emit an electrical pulse through the electrodes of the device. The trigger signal may include, for example, information relating to when the electrical pulse should be emitted, a pulse frequency, a pulse power or voltage, a pulse shape, and/or a pulse duration.

In some embodiments, the implantable device can also be operated to transmit information (i.e., uplink communication), which can be received by the interrogator, through the wireless communication system. In some embodiments, the wireless communication system is configured to actively generate a communication signal (e.g., ultrasonic waves or radiofrequency waves) that encode the information. In some embodiments, the wireless communication system is configured to transmit information encoded on backscatter waves (e.g., ultrasonic backscatter waves or RF backscatter waves). Backscatter communication provides a lower power method of transmitting information, which is particularly beneficial for small devices to minimize energy used. By way of example, the wireless communication system may include one or more ultrasonic transducers configured to receive ultrasonic waves and emit an ultrasonic backscatter, which can encode information transmitted by the implantable device. Current flows through the ultrasonic transducer, which can be modulated to encode the information. The current may be modulated directly, for example by passing the current through a sensor that modulates the current, or indirectly, for example by modulating the current using a modulation circuit based on a detected physiological signal.

In some embodiments, the information transmitted by the wireless communication system includes information unrelated to a detected physiological signal detected by the implantable device. For example, the information can include one or more of: information related to the status of the implantable device or a confirmation signal that confirms an electrical pulse was emitted, the power, frequency, voltage, duration, or other information related to an emitted electrical pulse, and/or an identification code for the implantable device. Optionally, the integrated circuit is configured to digitize the information, and the wireless communication system can transmit the digitized information.

The information wirelessly transmitted using the wireless communication system can be received by an interrogator. In some embodiments, the information is transmitted by being encoded in backscatter waves (e.g., ultrasonic backscatter or radiofrequency backscatter). The backscatter can be received by the interrogator, for example, and deciphered to determine the encoded information. Additional details about backscatter communication are provided herein, and additional examples are provided in WO 2018/009905; WO 2018/009908; WO 2018/009910; WO 2018/009911; WO 2018/009912; International Patent Application No. PCT/US2019/028381; International Patent Application No. PCT/US2019/028385; and International Patent Application No. PCT/2019/048647; each of which is incorporated herein by reference for all purposes. The information can be encoded by the integrated circuit using a modulation circuit. The modulation circuit is part of the wireless communication system, and can be operated by or contained within the integrated circuit.

An interrogator can transmit energy waves (e.g., ultrasonic waves or radiofrequency waves), which are received by the wireless communication system of the device to generate an electrical current flowing through the wireless communication system (e.g., to generate an electrical current flowing through the ultrasonic transducer or the radiofrequency antenna). The flowing current can then generate backscatter waves that are emitted by the wireless communication system. The modulation circuit can be configured to modulate the current flowing through the wireless communication system to encode the information. For example, the modulation circuit may be electrically connected to an ultrasonic transducer, which received ultrasonic waves from an interrogator. The current generated by the received ultrasonic waves can be modulated using the modulation circuit to encode the information, which results in ultrasonic backscatter waves emitted by the ultrasonic transducer to encode the information. A similar approach may be taken with a radiofrequency antenna that receives radiofrequency waves. The modulation circuit includes one or more switches, such as an on/off switch or a field-effect transistor (FET). An exemplary FET that can be used with some embodiments of the implantable device is a metal-oxide-semiconductor field-effect transistor (MOSFET). The modulation circuit can alter the impedance of a current flowing through the wireless communication system, and variation in current flowing through the wireless communication system encodes the information. In some embodiments, information encoded in the backscatter waves includes information related to an electrophysiological signal transmitted by the nerve, an electrical pulse emitted by the implantable device, or a physiological signal detected by a sensor of the implantable device. In some embodiments, information encoded in the backscatter waves includes a unique identifier for the implantable device. This can be useful, for example, to ensure the interrogator is in communication with the correct implantable device when a plurality of implantable devices is implanted in the subject. In some embodiments, the information encoded in the backscatter waves includes a verification signal that verifies an electrical pulse was emitted by the implantable device. In some embodiments, the information encoded in the backscatter waves includes an amount of energy stored or a voltage in the energy storage circuit (or one or more capacitors in the energy storage circuit). In some embodiments, the information encoded in the backscatter waves includes a detected impedance. Changes in the impedance measurement can identify scarring tissue or degradation of the electrodes over time.

In some embodiments, the modulation circuit is operated using a digital circuit or a mixed-signal integrated circuit (which may be part of the integrated circuit), which can actively encode the information in a digitized or analog signal. The digital circuit or mixed-signal integrated circuit may include a memory and one or more circuit blocks, systems, or processors for operating the implantable device. These systems can include, for example, an onboard microcontroller or processor, a finite state machine implementation, or digital circuits capable of executing one or more programs stored on the implant or provided via ultrasonic communication between interrogator and implantable device. In some embodiments, the digital circuit or a mixed-signal integrated circuit includes an analog-to-digital converter (ADC), which can convert analog signal encoded in the ultrasonic waves emitted from the interrogator so that the signal can be processed by the digital circuit or the mixed-signal integrated circuit. The digital circuit or mixed-signal integrated circuit can also operate the power circuit, for example to generate the electrical pulse to stimulate the tissue. In some embodiments, the digital circuit or the mixed-signal integrated circuit receives the trigger signal encoded in the ultrasonic waves transmitted by the interrogator, and operates the power circuit to discharge the electrical pulse in response to the trigger signal.

In some embodiments, the wireless communication system includes one or more ultrasonic transducers, such as one, two, or three or more ultrasonic transducers. In some embodiments, the wireless communication system includes a first ultrasonic transducer having a first polarization axis and a second ultrasonic transducer having a second polarization axis, wherein the second ultrasonic transducer is positioned so that the second polarization axis is orthogonal to the first polarization axis, and wherein the first ultrasonic transducer and the second ultrasonic transducer are configured to receive ultrasonic waves that power the device and emit an ultrasonic backscatter. In some embodiments, the wireless communication system includes a first ultrasonic transducer having a first polarization axis, a second ultrasonic transducer having a second polarization axis, and a third ultrasonic transducer having a third polarization axis, wherein the second ultrasonic transducer is positioned so that the second polarization axis is orthogonal to the first polarization axis and the third polarization axis, wherein the third ultrasonic transducer is positioned so that the third polarization axis is orthogonal to the first polarization and the second polarization axis, and wherein the first ultrasonic transducer and the second ultrasonic transducer are configured to receive ultrasonic waves that power the device and emit an ultrasonic backscatter. FIG. 2 shows a board assembly for a body of a device that includes two orthogonally positioned ultrasonic transducers. The board assembly includes a circuit board 202, such as a printed circuit board, and an integrated circuit 204, which a power circuit that includes a capacitor 206. The body further includes a first ultrasonic transducer 208 electrically connected to the integrated circuit 204, and a second ultrasonic transducer 210 electrically connected to the integrated circuit 204. The first ultrasonic transducer 208 includes a first polarization axis 212, and the second ultrasonic transducer 210 includes a second polarization axis 214. The first ultrasonic transducer 208 and the second ultrasonic transducer are positioned such that the first polarization axis 212 is orthogonal to the second polarization axis 214.

The ultrasonic transducer, if included in the wireless communication system, can be a micro-machined ultrasonic transducer, such as a capacitive micro-machined ultrasonic transducer (CMUT) or a piezoelectric micro-machined ultrasonic transducer (PMUT), or can be a bulk piezoelectric transducer. Bulk piezoelectric transducers can be any natural or synthetic material, such as a crystal, ceramic, or polymer. Exemplary bulk piezoelectric transducer materials include barium titanate (BaTiO3), lead zirconate titanate (PZT), zinc oxide (ZO), aluminum nitride (AlN), quartz, berlinite (AlPO4), topaz, langasite (La3Ga5SiO14), gallium orthophosphate (GaPO4), lithium niobate (LiNbO3), lithium tantalite (LiTaO3), potassium niobate (KNbO3), sodium tungstate (Na2WO3), bismuth ferrite (BiFeO3), polyvinylidene (di)fluoride (PVDF), and lead magnesium niobate-lead titanate (PMN-PT).

In some embodiments, the bulk piezoelectric transducer is approximately cubic (i.e., an aspect ratio of about 1:1:1 (length:width:height). In some embodiments, the piezoelectric transducer is plate-like, with an aspect ratio of about 5:5:1 or greater in either the length or width aspect, such as about 7:5:1 or greater, or about 10:10:1 or greater. In some embodiments, the bulk piezoelectric transducer is long and narrow, with an aspect ratio of about 3:1:1 or greater, and where the longest dimension is aligned to the direction of the ultrasonic backscatter waves (i.e., the polarization axis). In some embodiments, one dimension of the bulk piezoelectric transducer is equal to one half of the wavelength (λ) corresponding to the drive frequency or resonant frequency of the transducer. At the resonant frequency, the ultrasound wave impinging on either the face of the transducer will undergo a 180° phase shift to reach the opposite phase, causing the largest displacement between the two faces. In some embodiments, the height of the piezoelectric transducer is about 10 μm to about 1000 μm (such as about 40 μm to about 400 μm, about 100 μm to about 250 μm, about 250 μm to about 500 μm, or about 500 μm to about 1000 μm). In some embodiments, the height of the piezoelectric transducer is about 5 mm or less (such as about 4 mm or less, about 3 mm or less, about 2 mm or less, about 1 mm or less, about 500 μm or less, about 400 μm or less, 250 μm or less, about 100 μm or less, or about 40 μm or less). In some embodiments, the height of the piezoelectric transducer is about 20 μm or more (such as about 40 μm or more, about 100 μm or more, about 250 μm or more, about 400 μm or more, about 500 μm or more, about 1 mm or more, about 2 mm or more, about 3 mm or more, or about 4 mm or more) in length. In some embodiments, the ultrasonic transducer has a length of about 5 mm or less such as about 4 mm or less, about 3 mm or less, about 2 mm or less, about 1 mm or less, about 500 μm or less, about 400 μm or less, 250 μm or less, about 100 μm or less, or about 40 μm or less) in the longest dimension. In some embodiments, the ultrasonic transducer has a length of about 20 μm or more (such as about 40 μm or more, about 100 μm or more, about 250 μm or more, about 400 μm or more, about 500 μm or more, about 1 mm or more, about 2 mm or more, about 3 mm or more, or about 4 mm or more) in the longest dimension.

The ultrasonic transducer, if included in the wireless communication system, can be connected two electrodes to allow electrical communication with the integrated circuit. The first electrode is attached to a first face of the transducer and the second electrode is attached to a second face of the transducer, wherein the first face and the second face are opposite sides of the transducer along one dimension. In some embodiments, the electrodes comprise silver, gold, platinum, platinum-black, poly(3,4-ethylenedioxythiophene (PEDOT), a conductive polymer (such as conductive PDMS or polyimide), or nickel. In some embodiments, the axis between the electrodes of the transducer is orthogonal to the motion of the transducer.

The implantable device may be configured to wirelessly receive energy and convert the energy into an electrical energy, which may be used to power the device. The wireless communication system may be used to wireless receive the energy, or a separate system may be configured to receive the energy. For example, an ultrasonic transducer (which may be an ultrasonic transducer contained within the wireless communication system or a different ultrasonic transducer) can be configured to receive ultrasonic waves and convert energy from the ultrasonic waves into an electrical energy. In some embodiments, an RF antenna (which may be an RF antenna contained within the wireless communication system or a different RF antenna) is configured to receive RF waves and convert the energy from the RF waves into an electrical energy. The electrical energy is transmitted to the integrated circuit to power the device. The electrical energy may power the device directly, or the integrated circuit may operate a power circuit to store the energy for later use.

In some embodiments, the integrated circuit includes a power circuit, which can include an energy storage circuit. The energy storage circuit may include a battery, or an alternative energy storage device such as one or more capacitors. The implantable device is preferably batteryless, and may instead rely on one or more capacitors. By way of example, energy from ultrasonic waves or radiofrequency waves received by the implantable device (for example, through the wireless communication system) is converted into a current, and can be stored in the energy storage circuit. The energy can be used to operate the implantable device, such as providing power to the digital circuit, the modulation circuit, or one or more amplifiers, or can be used to generate the electrical pulse used to stimulate the tissue. In some embodiments, the power circuit further includes, for example, a rectifier and/or a charge pump.

The integrated circuit may be configured to operate the two or more electrodes of the device configured to detect an electrophysiological signal transmitted by a nerve or emit an electrical pulse to the nerve, and at least one of the electrodes is included on the nerve cuff. The electrodes may be positioned on the nerve cuff, the body of the device, or both (e.g., one or more electrodes may be on the body of the device and one or more electrodes may be on the nerve cuff). In some embodiments, the housing of the body operates as an electrode. For example, the device may include one or more working electrodes on the nerve cuff, and the housing may be configured as a counter electrode. Accordingly, in some embodiments, the housing of the device is electrically connected to the integrated circuit. The one or more electrodes on the nerve cuff are electrically connected to the integrated circuit, for example through one or more feedthroughs.

In some embodiments, the implantable device includes one or more sensors configured to detect a physiological signal. The sensor(s) may be, for example, included as part of the body of the device or on the nerve cuff. The sensors are configured to detect a physiological signal, such as temperature, oxygen concentration, pH, an analyte (such as glucose), strain, or pressure. Variation in the physiological signal modulates impedance, which in turn modulates current flowing through a detection circuit electrically connected to or part of the integrated circuit. The implantable device may comprise one or more (such as 2, 3, 4, 5 or more) sensors, which may detect the same physiological signal or different physiological signals. In some embodiments, the implantable device comprises 10, 9, 8, 7, 6 or 5 or fewer sensors). For example, in some embodiments, the implantable device comprises a first sensor configured to detect temperature and a second sensor configured to detect oxygen. Changes in both physiological signals can be encoded in the backscatter waves emitted by the wireless communication system, which can be deciphered by an external computing system (such as the interrogator).

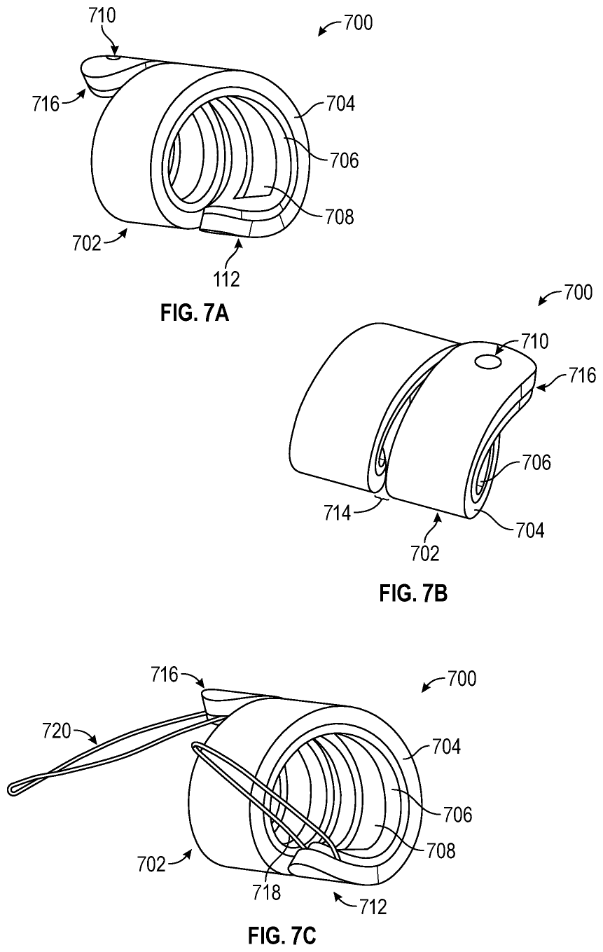

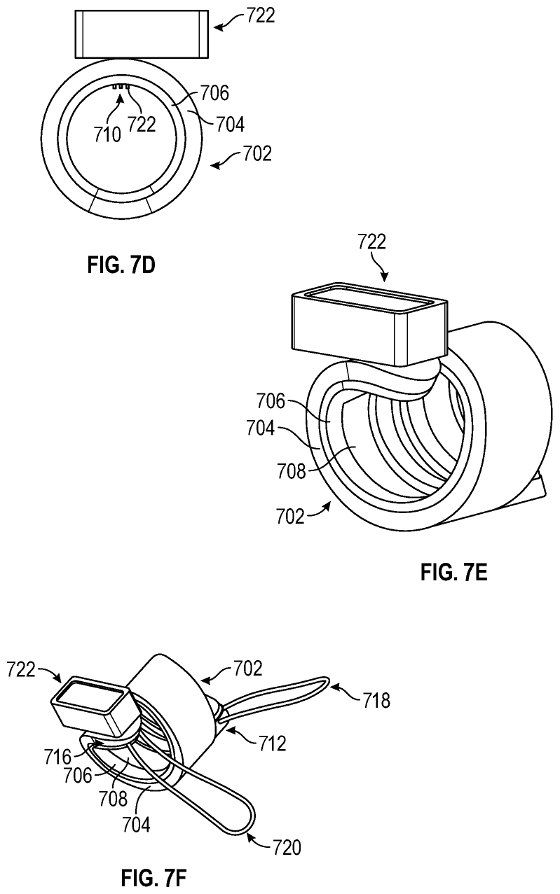

The body of the implantable device is attached to the nerve cuff, for example on the outer surface of the helical nerve cuff. In some embodiments, the body is attached to an end of the nerve cuff, or at a middle portion of the nerve cuff. Optionally, a handle portion may be attached to the nerve cuff, and may be attached at a position proximal to the body. In some embodiments, the implantable device includes a handle portion attached to the helical nerve cuff at a position proximal to the body attached to the nerve cuff, and a second handle portion attached to the nerve cuff at a distal position, such as at an end of the nerve cuff. Examples of an implantable device body attached to a helical nerve cuff is shown in FIGS. 7D, 7E, 7F, 8C, and 9C. In some embodiments, a handle portion is attached to the body of the implantable device.

The body of the of the implantable device may be attached to nerve cuff through an adhesive (e.g., an epoxy, glue, cement, solder, or other binder), one or more fasteners (e.g., a staple, screw, bolt, clap, rivet, pin, rod, etc.), or any other suitable means to securely attach the body to the nerve cuff to ensure that it does not become separated from the nerve cuff after implantation. FIG. 3 shows an exemplary body 302 attached to a nerve cuff 304 using fasteners (306 and 308). In some embodiment, the body has an elongated shape, and one end of the body (i.e., the attachment end) is attached to the nerve cuff, and the opposite end (i.e., an extension end) extends from the nerve cuff (see, for example, the body attached to the nerve cuff in FIG. 7E). The body is directly attached to the outer surface of the nerve cuff (i.e., without any interceding lead between body and the nerve cuff).

The body can include a housing, which can include a base, one or more sidewalls, and a top. The housing is optionally made from an electrically conductive material and may be configured as one of the one or more electrodes of the implantable device configured to detect an electrophysiological signal transmitted by a nerve or emit an electrical pulse to the nerve. For example, the housing of the body may be configured as a counter electrode, and one or more electrodes on the nerve cuff may be configured as an operating electrode. The housing is made from a bioinert material, such as a bioinert metal (e.g., steel or titanium) or a bioinert ceramic (e.g., titania or alumina). The housing is preferably hermetically sealed, which prevents body fluids from entering the body.

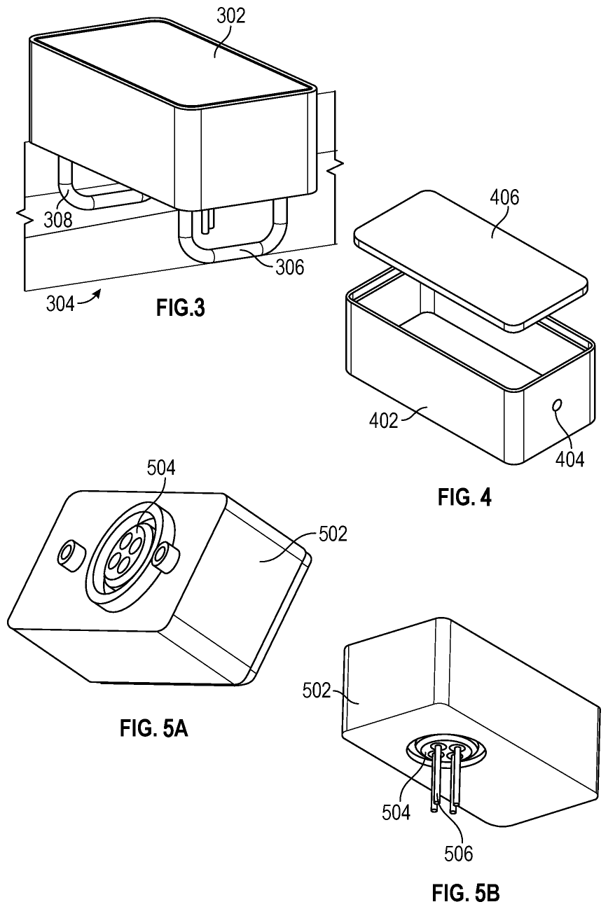

Referring to FIG. 4, an acoustic window 406 can be included in the housing 402 of the body, for example on the top of the housing. An acoustic window is a thinner material (such as a foil) that allows acoustic waves to penetrate the housing so that they may be received by one or more ultrasonic transducers within the body of the implantable device. In some embodiments, the housing (or the acoustic window of the housing) may be thin to allow ultrasonic waves to penetrate through the housing is about 100 micormeters (μm) or less in thickness, such as about 75 μm or less, about 50 μm or less, about 25 μm or less, about 15 μm or less, or about 10 μm or less. In some embodiments, the thickness of the housing (or the acoustic window of the housing) is about 5 μm to about 10 μm, about 10 μm to about 15 μm, about 15 μm to about 25 μm, about 25 μm to about 50 μm, about 50 μm to about 75 μm, or about 75 μm to about 100 μm in thickness.

The housing 402 may be filled with an acoustically conductive material, such as a polymer or oil (such as a silicone oil). The material can fill empty space within the housing to reduce acoustic impedance mismatch between the tissue outside of the housing and within the housing. Accordingly, the body of the device is preferably void of air or vacuum. A port 404 can be included on the housing, for example on the sidewall of the housing (see FIG. 4), to allow the housing to be filled with the acoustically conductive material. Once the housing is filled with the material, the port can be sealed to avoid leakage of the material after implantation.

The housing of the implantable device is relatively small, which allows for comfortable and long-term implantation while limiting tissue inflammation that is often associated with implantable devices. In some embodiments, the longest dimension of the housing of the device is about 8 mm or less, about 7 mm or less, about 6 m or less, about 5 mm or less, about 4 mm or less, about 3 mm or less, about 2 mm or less, about 1 mm or less, about 0.5 mm or less, about 0.3 mm or less, about 0.1 mm or less in length. In some embodiments, the longest dimension of the housing of the device is about 0.05 mm or longer, about 0.1 mm or longer, about 0.3 mm or longer, about 0.5 mm or longer, about 1 mm or longer, about 2 mm or longer, about 3 mm or longer, about 4 mm or longer, about 5 mm or longer, about 6 mm or longer, or about 7 mm or longer in the longest dimension of the device. In some embodiments, the longest dimension of the housing of the device is about 0.3 mm to about 8 mm in length, about 1 mm to about 7 mm in length, about 2 mm to about 6 mm in length, or about 3 mm to about 5 mm in length. In some embodiments, the housing of the implantable device has a volume of about 10 mm³ or less (such as about 8 mm³ or less, 6 mm³ or less, 4 mm³ or less, or 3 mm³ or less). In some embodiments, the housing of the implantable device has a volume of about 0.5 mm³ to about 8 mm³, about 1 mm³ to about 7 mm³, about 2 mm³ to about 6 mm³, or about 3 mm³ to about 5 mm³.

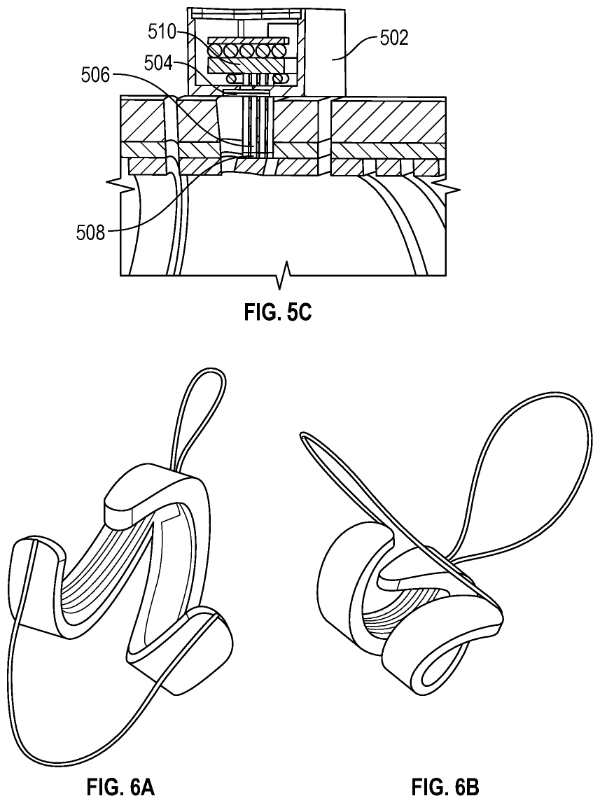

The housing (such as the bottom of the housing) can include a feedthrough port, which may be aligned with the feedthrough port of the nerve cuff. A feedthrough can electrically connect the one or more electrodes of the nerve cuff to components of the body within the housing. For example, the feedthrough may be electrically connected to an integrated circuit and/or the wireless communication system of the device body. FIG. 5A shows a housing 502 with a feedthrough port 504, and FIG. 5B shows the housing with the feedthrough 506 positioned to electrically connect the body components to one or more electrodes of the nerve cuff. FIG. 5C shows a cross-sectional view of an exemplary device, wherein the feedthrough 506 electronically connects electrodes 508 on the nerve cuff to the electronic circuitry 510 (integrated circuit, wireless communication system, etc.) positioned within the body housing 502. The feedthroughs may be, for example, a metal (such as a metal comprising silver, copper, gold, platinum, platinum-black, or nickel) sapphire, or a conductive ceramic (for example indium tin oxide (ITO)). The electrodes may be connected to the feedthrough using any suitable means, such as soldering, laser welding, or crimping the feedthrough to the electrodes.

In some embodiments, the implantable device is implanted in a subject. The subject can be for example, a mammal. In some embodiments, the subject is a human, dog, cat, horse, cow, pig, sheep, goat, monkey, or a rodent (such as a rat or mouse). The nerve cuff may be configured to at least partially wrap around the splanchnic nerve within any of these animals, or others.

### Implantable Device Nerve Cuff

In some embodiments, the implantable device includes a nerve cuff sized and configured to attach the device to the thoracic splanchnic nerve and position at least one of the one or more electrodes in electrical communication with the greater splanchnic nerve. In some embodiments, the thoracic splanchnic nerve is the greater splanchnic nerve, the lesser splanchnic nerve, or the leas splanchnic nerve. In some embodiments, the greater splanchnic nerve cuff is a helical nerve cuff.

The nerve cuff holds the implantable device in place on the thoracic splanchnic nerve. In some embodiments, the nerve cuff allows for some rotational movement of the implantable device on the nerve. In some embodiments, the nerve cuff grips the thoracic splanchnic nerve by exerting an inward pressure on the nerve. The amount of inward pressure exerted by the nerve cuff can be determined based on the size and curvature of the nerve cuff, as well as by the spring constant of the nerve cuff. The inward pressure should be sufficient to hold the implantable device in place while the tissue heals after insertion, but not so high that the epineurium or vascular walls that contact the legs are damaged. In some embodiments, the inward pressure on the nerve is about 1 MPa or less (such as about 0.7 MPa or less, about 0.5 MPa or less, or about 0.3 MPa or less). In some embodiments, the inward pressure on the nerve is about 0.1 MPa to about 1 MPa (such as about 0.1 MPa to about 0.3 MPa, about 0.3 MPa to about 0.5 MPa, about 0.5 MPa to about 0.7 MPa, or about 0.7 MPa to about 1 MPa).

The nerve cuff includes a helical substrate configured to at least partially wrap around a filamentous tissue comprising a nerve, and one or more electrodes positioned along the length of the substrate. The nerve cuff may optionally include one or more handle portions, for example a handle portion attached to an end of the substrate. In some embodiments, the nerve cuff is a helical nerve cuff.

The inner diameter of the nerve cuff may be selected based on the diameter of the filamentous tissue, which may different depending on the species of the subject or other anatomical differences within the subject (e.g., the size of the nerve within the specific subject. By way of example, the inner diameter may be between about 0.5 mm and about 5 mm in diameter (such as between about 0.5 mm and about 1 mm, about 1 mm and about 2 mm, about 2 mm and about 3 mm, about 3 mm and about 4 mm, or about 4 mm and about 5 mm in diameter).

The nerve cuff may be configured to wrap around the nerve by at least one revolution. For example, the nerve cuff may wrap around the nerve by about 1 to about 4 revolutions, such as about 1 to about 1.3 revolutions, about 1.3 to about 1.7 revolutions, about 1.7 to about 2 revolutions, about 2 to about 2.5 revolutions, about 2.5 to about 3 revolutions, or about 3 to about 4 revolutions. In some embodiments, the nerve cuff is configured to wrap around the nerve by about 1.5 revolutions.

In some embodiments, the substrate of the nerve cuff is an elongated material wound into a helical shape. The helical substrate may have a substantially flat inner surface and/or a substantially flat outer surface. The width of the substrate may be substantially uniform, with optionally tapered or rounded ends. The width of the substrates define edges, and the edges may or may not contact each other as the substrate winds in the helical shape when the nerve cuff is in a relaxed position. For example, in some embodiments, a gap may or may not separate the revolutions of the substrate. In some embodiments, the substrate has a width that defines an inner surface, a first edge of the substrate, and a second edge of the substrate, and wherein at least a portion of the first edge contacts at least a portion of the second edge when the nerve cuff is in a relaxed position. In some embodiments, the substrate has a width that defines an inner surface, a first edge of the substrate, and a second edge of the substrate, and wherein the first edge does not contact the second edge when the nerve cuff is in a relaxed position.

The substrate of the nerve cuff is made from an insulating material, which may be a biocompatible and/or elastomeric material. Exemplary substrate materials include, but are not limited to, silicone, silicone rubber, polydimethylsioloxane (PDMS), a urethane polymer, a poly(p-xylylene) polymer (such as a poly(p-xylylene) polymer sold under the tradename PARYLENE®), or a polyimide.

In some embodiments, the substrate of the nerve cuff may include two or more layers, which may be of the same material or of different materials. The layers can included an inner layer that forms the inner surface of the nerve cuff and contacts the fibrous tissue, and an outer layer that forms that forms the outer surface of the nerve cuff. An electrically conductive material may be positioned between the inner and outer layers, which can form the electrodes of the nerve cuff. For example, the inner layer can include one or more opening on the inner surface to expose the electrically conductive material, which defines the electrodes. The separate inner and outer layers can further define the helical shape of the substrate. For example, the inner layer may be under higher tensile force than the outer layer when the helical nerve cuff is in a flexed configuration, which forces the substrate to curl inwards when the helical nerve cuff is in a relaxed configuration.

The width of the nerve cuff can depend on the on the length of nerve cuff (i.e., the maximal distance along an axis running through the center of the helix between the ends of the nerve cuff), the number of revolutions of the substrate, and size of a gap between substrate revolutions (if any). In some embodiments, the length of the nerve cuff is about 4 mm to about 20 mm (such as about 4 mm to about 7 mm, about 7 mm to about 10 mm, about 10 mm to about 13 mm, about 13 mm to about 16 mm, or about 16 mm to about 20 mm). In some embodiments, the width (or the inner width) of the substrate is about 2 mm to about 8 mm (such as about 2 mm to about 4 mm, about 4 mm to about 6 mm, or about 6 mm to about 8 mm).

The nerve cuff may be flexible, which allows for manipulation of the nerve cuff upon implantation. For example, in some embodiments, the helical nerve cuff can be configured in a flexed position by at least partially unwinding the helical nerve cuff, and a relaxed position with the helical nerve cuff in a helical configuration. FIG. 6A shows an exemplary helical nerve cuff in a flexed position, wherein both the right-handed helical portion and the left-handed helical portion of the nerve cuff are partially unwound by pulling a first handle portion and a second handle portion which are joined together and attached to either end of the right-handed helical portion and the left-handed helical portion in one direction, and pulling a third handle portion attached to a joining member in the opposite direction. FIG. 6B shows the same helical nerve cuff shown in FIG. 6A in a relaxed position.

The nerve cuff may include a right-handed helical portion, a left-handed helical portion, or both a right-handed helical portion a left-handed helical portion. For example, in some embodiments, the nerve cuff may include a right-handed helical portion joined to a left-handed helical portion, either directly or through a connecting member (which may be linear, curved, or hinged).

The one or more electrodes of the nerve cuff may be positioned on the inner surface of the nerve cuff substrate, and may be uncoated or coated with an electrically conductive material (e.g., electroplated with a poly(3,4-ethylenedioxythiophene) (PEDOT) polymer or other electrically conductive polymer or a metal to improve electrical characteristics of the electrode). In some embodiments, one or more of the electrodes are point electrodes. In some embodiments, one or more of the electrodes may be elongated, and may be positioned, for example, along the length of the substrate. The electrodes may terminate before the end of the substrate, at the end of the substrate, or beyond the end of the substrate. The one or more electrodes may be connected to a feed through on the nerve cuff, which allows the electrodes to be electrically connected to the outer surface of the substrate or a body attached to the outer surface of the nerve cuff.

The nerve cuff includes one or more electrodes, such as 1, 2, 3, 4, 5, 6, 7, 8, 9, 10 or more electrodes. In some embodiments, one or more of the electrodes are configured to emit an electrical pulse to the nerve. In some embodiments, the nerve cuff includes 1, 2, 3, 4, 5, 6, 7, 8, 9, 10 or more electrodes configured to emit an electrical pulse to the nerve. In some embodiments, one or more of the electrodes are configured to detect an electrophysiological signal transmitted by the nerve. In some embodiments, the nerve cuff includes 1, 2, 3, 4, 5, 6, 7, 8, 9, 10 or more electrodes configured to detect an electrophysiological signal transmitted by the nerve. In some embodiments, one or more electrodes are configured to emit an electrical pulse to the nerve, and one or more of the electrodes are configured to detect an electrophysiological signal transmitted by the nerve. In some embodiments, an electrode configured to emit an electrical pulse is wider than an electrode configured to detect an electrophysiological signal. Electrodes of the nerve cuff (having more than one electrode) may be positioned along the length of the nerve cuff alongside each other, or in different directions.

The optional handle portion is configured to be grasped by a surgical grasping tool (e.g., forceps, hook, or other grasping or gripping instrument), and may be useful for manipulating the nerve cuff during implantation. The handle portion may extend from or be partially embedded within the substrate, and may be more flexible and/or thinner than the substrate to facilitate grasping of the handle portion and manipulation of the nerve cuff. The handle portion may include a loop, for example within the handle portion or by forming a loop by either end of the handle portion being attached to the substrate. In some embodiments, the handle portion comprises a flexible filament (such as a thread, string, cord, suture, or wire), which is optionally biodegradable once implanted within the subject. In some embodiments, the handle portion comprises a bioabsorbable material, such as polyglycolide, polydioxanone, polycaprolactone, or copolymers thereof.

The optional handle portion may be attached to the nerve cuff proximal to the end of the nerve cuff (e.g., at the tip of the nerve cuff). The nerve cuff optionally includes more than one handle portion. For example, the substrate may include an additional handle portion proximal to the opposite end of the substrate and/or an additional handle portion proximal to a middle portion of the substrate. If the nerve cuff is attached to a body, as further discussed herein, the one of the handle portions may be proximal to the body or distal to the body. By way of example, in some embodiments, the body is attached proximal to a first end of the nerve cuff, and the handle portion is attached proximal to the second end of the helical nerve cuff. In some embodiments, the body is attached proximal to a first end of the nerve cuff, and the handle portion is attached proximal to the first end of the nerve cuff. In some embodiments, a body is attached proximal to a first end of the nerve cuff, and a first handle portion is attached proximal to the body and a second handle portion is attached proximal to a second end of the nerve cuff. In some embodiments, the body is attached to a middle portion of the nerve cuff, a first handle portion is attached proximal to a first end of the nerve cuff, a second handle portion is attached proximal to the body, and optionally a third handle portion is attached proximal to the second end of the nerve cuff.

Optionally, two or more handle portions attached to the nerve cuff are joined together. For example, a first handle portion includes a first end attached proximal to a first end of the helical nerve cuff, a second handle portion includes a first end attached proximal to a second end of the nerve cuff, and the second end of the first handle portion and the second end of the second handle portion are joined together.

FIG. 7A illustrates an exemplary helical nerve cuff, which may optionally be part of the implantable device described herein. FIG. 7B shows the nerve cuff illustrated in FIG. 7A from a different angle. The nerve cuff 700 includes a helical substrate 702 that includes an outer layer 704 and an inner layer 706. The nerve cuff is configured to wrap around the nerve by about 1.5 revolutions, and a gap 714 separates substrate revolutions. The substrate 702 is configured as a left-handed helix, although an embodiment with a right-handed helical substrate is also contemplated. An elongate electrode 708 is positioned on the inner surface of the helical substrate 702. The elongated electrode 708 spans from a feedthrough port 710, and terminates at a position before the end 712 of the helical substrate 702. The electrode 708 is between the outer layer 704 and the inner layer 706, and the inner layer 106 includes an elongated cutout that exposes the electrode 708 to the inner surface of the nerve cuff 700. In an alternative embodiment, the electrode is positioned on top of the inner layer 706. FIG. 7D and FIG. 7E show the helical nerve cuff of FIG. 7A and FIG. 7B attached to a body having a housing 722. The housing 722 is attached to the outer surface of the helical nerve cuff substrate 702. A feedthrough 724 passes through the feedthrough port 710 at electrically connects the elongated electrode 708 to the body.

FIG. 7C illustrates an exemplary helical nerve cuff similar to the nerve cuff illustrated in FIG. 7A and FIG. 7B, but further includes a first handle portion 718 attached to the helical substrate 702 proximal to a first end 712 of the substrate 702, and a second handle portion 720 attached to the helical substrate 702 proximal to a second end 716 of the substrate 702. The first handle portion 718 and the second handle portion 720 are each flexible filaments that form a loop, with each end of the filament attached to the substrate 702. The ends of the filament are embedded within the substrate 702 between the inner layer 706 and the outer layer 704. FIG. 7F shows the helical nerve cuff of FIG. 7C attached to a body having a housing 722. The housing 722 is attached to the outer surface of the helical nerve cuff substrate 702.

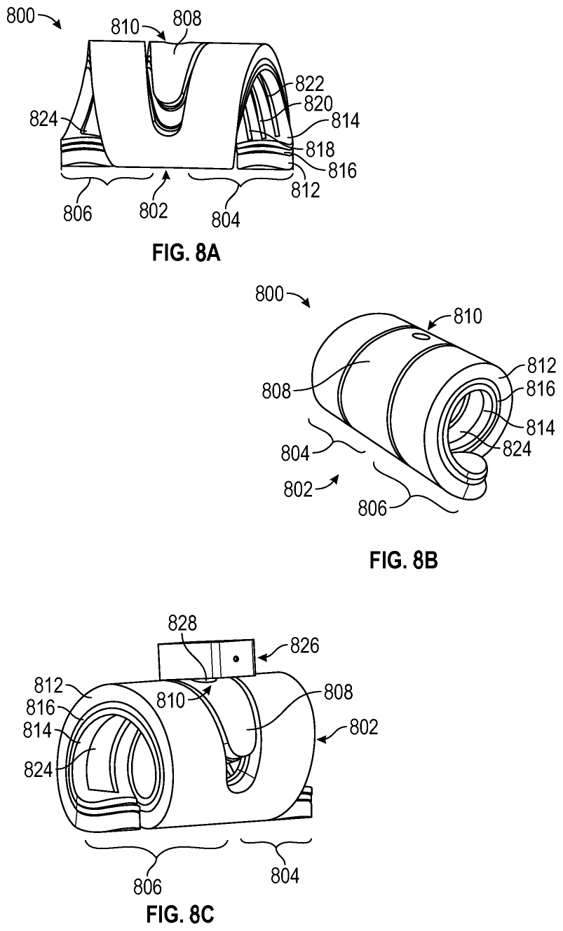

FIG. 8A and FIG. 8B illustrate front and back perspectives, respectively, of another embodiment of a helical nerve cuff 800. The nerve cuff 800 includes a substrate 802 with a left-handed helical segment 804 and a right-handed helical segment 806 joined together through a connecting member 808. The connecting member 808 of the illustrated nerve cuff 800 is a curved and elongated portion of the substrate 802 that makes slightly less than one full rotation around the nerve. A feedthrough port 810 is positioned along the connecting member 808, which allows a body to be electrically connected to electrodes positioned on the inner surface of the substrate. The substrate 802 includes an outer layer 812 and an inner layer 814, which sandwiches and electrically conductive middle layer 816 between the outer layer 812 and the inner layer 814. The helical nerve cuff includes three parallel elongated electrodes (818, 820, and 822) configured to detect an electrophysiological signal transmitted by a nerve on the inner surface of the substrate 802 at the left-handed helical segment 204, and a fourth elongated electrode 824 configured to emit an electrical pulse to the nerve on the inner surface of the substrate 802 at the right-handed helical segment 206. The electrodes are defined by an opening in the inner layer 814. In the illustrated embodiment, the fourth elongated electrode 824 is wider than the electrodes 818, 820, and 822. FIG. 8C shows the helical nerve cuff of FIG. 8A and FIG. 8B attached to a body having a housing 826. The housing 826 is attached to the outer surface of the helical nerve cuff substrate 802 at the connecting member 808. A feedthrough 828 passes through the feedthrough port 810 at electrically connects the electrodes 818, 820, 822, and 824 to the body.

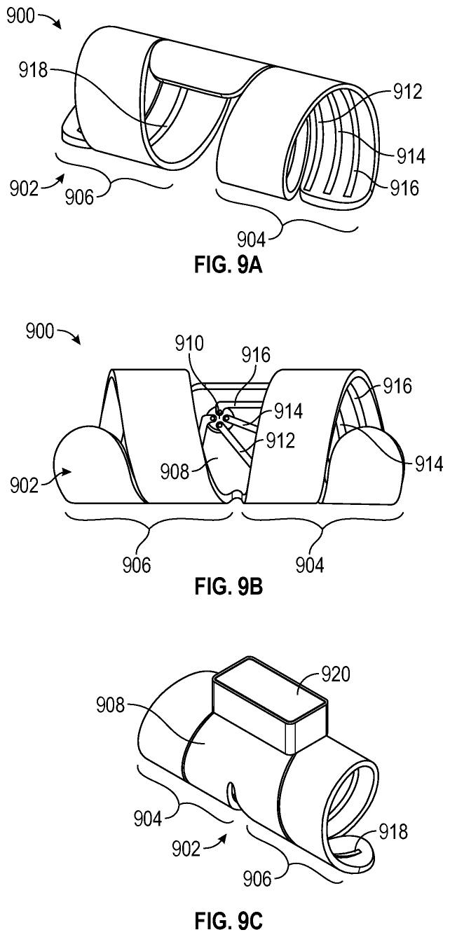

FIG. 9A and FIG. 9B illustrate front and bottom perspectives, respectively, of another embodiment of a helical nerve cuff 900. The nerve cuff 900 includes a substrate 902 with a left-handed helical segment 904 and a right-handed helical segment 906 joined together through a connecting member 908. The connecting member 908 of the illustrated nerve cuff 900 is a curved and elongated portion of the substrate 902, which is shorter than the connecting member of the nerve cuff illustrated in FIG. 8A and FIG. 8B. A feedthrough port 910 is positioned along the connecting member 908, which allows a body to be electrically connected to electrodes positioned on the inner surface of the substrate. The substrate 302 of the illustrated never cuff 900 includes a single layer, with electrodes positioned along the inner surface of the substrate 902. The helical nerve cuff includes three elongated electrodes (912, 914, and 916) on the inner surface of the substrate 902 at the left-handed helical segment 904, and a fourth elongated electrode 918 on the inner surface of the substrate 902 at the right-handed helical segment 906. FIG. 9C shows the helical nerve cuff of FIG. 9A and FIG. 9B attached to a body having a housing 920. The housing 920 is attached to the outer surface of the helical nerve cuff substrate 902.

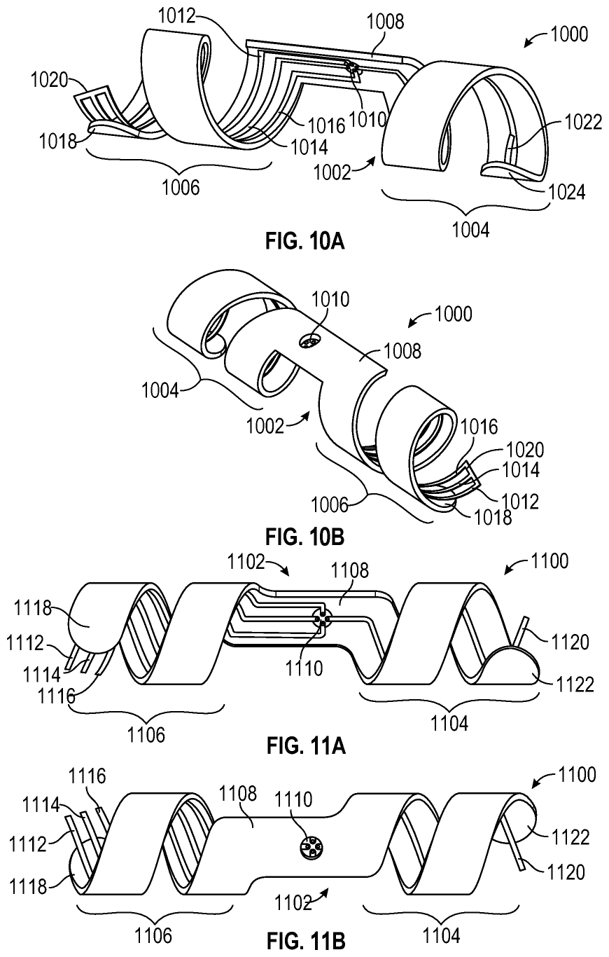

FIG. 10A and FIG. 10B illustrate bottom and top perspectives, respectively, of another embodiment of a helical nerve cuff 1000. The nerve cuff 1000 includes a substrate 1002 with a left-handed helical segment 1004 and a right-handed helical segment 1006 joined together through a connecting member 1008. The connecting member 1008 of the illustrated nerve cuff 1000 is an elongated and linear connecting member. A feedthrough port 1010 is positioned along the connecting member 1008, which allows a body to be electrically connected to electrodes positioned on the inner surface of the substrate. The substrate 1002 of the illustrated never cuff 1000 includes a single layer, with electrodes positioned along the inner surface of the substrate 1002. The helical nerve cuff includes three parallel elongated electrodes (1012, 1014, and 1016) on the inner surface of the substrate 1002 at the left-banded helical segment 1004, and extend beyond the end 1018 of the nerve cuff 1000. In the illustrated embodiment, the electrodes 1012, 1014, and 1016 which are joined together at a joining end 1020. The nerve cuff further includes a fourth elongated electrode 1022 on the inner surface of the substrate 1002 at the right-handed helical segment 1006, which extends beyond the opposite end 1024 of the nerve cuff 1000.

FIG. 11A and FIG. 11B illustrate bottom and top perspectives, respectively, of another embodiment of a helical nerve cuff 1100. The nerve cuff 1100 includes a substrate 1102 with a first left-handed helical segment 1104 and a second left-handed helical segment 1106 joined together through a connecting member 1108. The connecting member 1108 of the illustrated nerve cuff 1100 is an elongated and linear connecting member. A feedthrough port 1110 is positioned along the connecting member 1108, which allows a body to be electrically connected to electrodes positioned on the inner surface of the substrate. The substrate 1102 of the illustrated never cuff 1100 includes a single layer, with electrodes positioned along the inner surface of the substrate 1102. The helical nerve cuff includes three parallel elongated electrodes (1112, 1114, and 1116) on the inner surface of the substrate 1102 at the first left-handed helical segment 1104, and extend beyond the end 1118 of the nerve cuff 1100. The nerve cuff further includes a fourth elongated electrode 1120 on the inner surface of the substrate 1102 at the second left-handed helical segment 1106, which extends beyond the opposite end 1122 of the nerve cuff 1100.

### Interrogator

A second device, such as an interrogator, can wirelessly communicate with one or more implantable devices using ultrasonic waves, which are used to power and/or operate the implantable device. For example, the interrogator can transmit ultrasonic waves that encode instructions for operating the device, such as a trigger signal that instructs the implantable device to emit an electrical pulse. The interrogator can further receive ultrasonic backscatter from the implantable device, which encodes information transmitted by the implantable device. The information may include, for example, information related to a detected electrophysiological pulse, an electrical pulse emitted by the implantable device, and/or a measured physiological signal. The interrogator includes one or more ultrasonic transducers, which can operate as an ultrasonic transmitter and/or an ultrasonic receiver (or as a transceiver, which can be configured to alternatively transmit or receive the ultrasonic waves). The one or more transducers can be arranged as a transducer array, and the interrogator can optionally include one or more transducer arrays. In some embodiments, the ultrasound transmitting function is separated from the ultrasound receiving function on separate devices. That is, optionally, the interrogator comprises a first device that transmits ultrasonic waves to the implantable device, and a second device that receives ultrasonic backscatter from the implantable device. In some embodiments, the transducers in the array can have regular spacing, irregular spacing, or be sparsely placed. In some embodiments the array is flexible. In some embodiments the array is planar, and in some embodiments the array is non-planar.

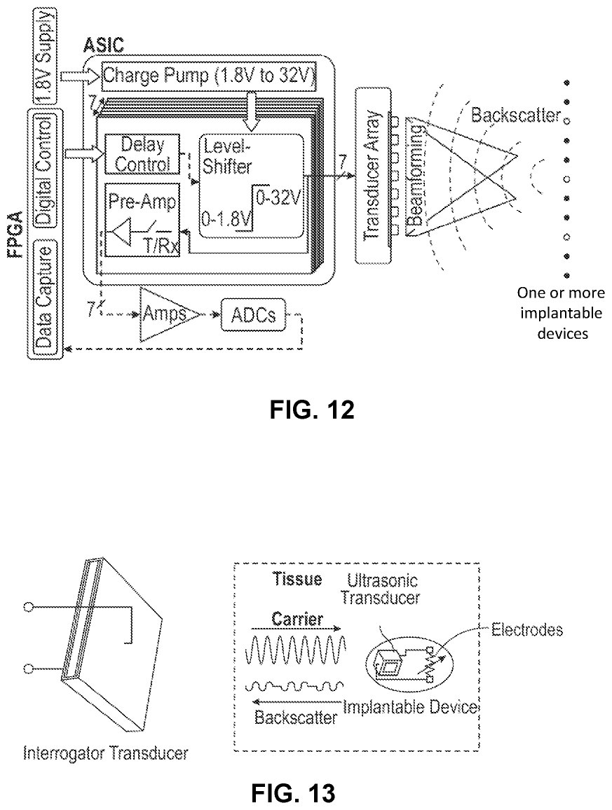

An exemplary interrogator is shown in FIG. 12. The illustrated interrogator shows a transducer array with a plurality of ultrasonic transducers. In some embodiments, the transducer array includes 1 or more, 2 or more, 3 or more, 5 or more, 7 or more, 10 or more, 15 or more, 20 or more, 25 or more, 50 or more, 100 or more 250 or more, 500 or more, 1000 or more, 2500 or more, 5000 or more, or 10,000 or more transducers. In some embodiments, the transducer array includes 100,000 or fewer, 50,000 or fewer, 25,000 or fewer, 10,000 or fewer, 5000 or fewer, 2500 or fewer, 1000 or fewer, 500 or fewer, 200 or fewer, 150 or fewer, 100 or fewer, 90 or fewer, 80 or fewer, 70 or fewer, 60 or fewer, 50 or fewer, 40 or fewer, 30 or fewer, 25 or fewer, 20 or fewer, 15 or fewer, 10 or fewer, 7 or fewer or 5 or fewer transducers. The transducer array can be, for example a chip comprising 50 or more ultrasonic transducer pixels.

The interrogator shown in FIG. 12 illustrates a single transducer array; however the interrogator can include 1 or more, 2 or more, or 3 or more separate arrays. In some embodiments, the interrogator includes 10 or fewer transducer arrays (such as 9, 8, 7, 6, 5, 4, 3, 2, or 1 transducer arrays). The separate arrays, for example, can be placed at different points of a subject, and can communicate to the same or different implantable devices. In some embodiments, the arrays are located on opposite sides of an implantable device. The interrogator can include an application specific integrated circuit (ASIC), which includes a channel for each transducer in the transducer array. In some embodiments, the channel includes a switch (indicated in FIG. 12 by “T/Rx”). The switch can alternatively configure the transducer connected to the channel to transmit ultrasonic waves or receive ultrasonic waves. The switch can isolate the ultrasound receiving circuit from the higher voltage ultrasound transmitting circuit.

In some embodiments, the transducer connected to the channel is configured only to receive or only to transmit ultrasonic waves, and the switch is optionally omitted from the channel. The channel can include a delay control, which operates to control the transmitted ultrasonic waves. The delay control can control, for example, the phase shift, time delay, pulse frequency and/or wave shape (including amplitude and wavelength). The delay control can be connected to a level shifter, which shifts input pulses from the delay control to a higher voltage used by the transducer to transmit the ultrasonic waves. In some embodiments, the data representing the wave shape and frequency for each channel can be stored in a ‘wave table’. This allows the transmit waveform on each channel to be different. Then, delay control and level shifters can be used to ‘stream’ out this data to the actual transmit signals to the transducer array.

In some embodiments, the transmit waveform for each channel can be produced directly by a high-speed serial output of a microcontroller or other digital system and sent to the transducer element through a level shifter or high-voltage amplifier. In some embodiments, the ASIC includes a charge pump (illustrated in FIG. 12) to convert a first voltage supplied to the ASIC to a higher second voltage, which is applied to the channel. The channels can be controlled by a controller, such as a digital controller, which operates the delay control.

In the ultrasound receiving circuit, the received ultrasonic waves are converted to current by the transducers (set in a receiving mode), which is transmitted to a data capture circuit. In some embodiments, an amplifier, an analog-to-digital converter (ADC), a variable-gain-amplifier, or a time-gain-controlled variable-gain-amplifier which compensates for tissue loss, and/or a band pass filter is included in the receiving circuit. The ASIC can draw power from a power supply, such as a battery (which is preferred for a wearable embodiment of the interrogator). In the embodiment illustrated in FIG. 12, a 1.8V supply is provided to the ASIC, which is increased by the charge pump to 32V, although any suitable voltage can be used. In some embodiments, the interrogator includes a processor and or a non-transitory computer readable memory. In some embodiments, the channel described above does not include a T/Rx switch but instead contains independent Tx (transmit) and Rx (receive) with a high-voltage Rx (receiver circuit) in the form of a low noise amplifier with good saturation recovery. In some embodiments, the T/Rx circuit includes a circulator. In some embodiments, the transducer array contains more transducer elements than processing channels in the interrogator transmit/receive circuitry, with a multiplexer choosing different sets of transmitting elements for each pulse. For example, 64 transmit receive channels connected via a 3:1 multiplexer to 192 physical transducer elements—with only 64 transducer elements active on a given pulse.

In some embodiments, the interrogator is implantable. In some embodiments, the interrogator is external (i.e., not implanted). By way of example, the external interrogator can be a wearable, which may be fixed to the body by a strap or adhesive. In another example, the external interrogator can be a wand, which may be held by a user (such as a healthcare professional). In some embodiments, the interrogator can be held to the body via suture, simple surface tension, a clothing-based fixation device such as a cloth wrap, a sleeve, an elastic band, or by sub-cutaneous fixation. The transducer or transducer array of the interrogator may be positioned separately from the rest of the transducer. For example, the transducer array can be fixed to the skin of a subject at a first location (such as proximal to one or more implanted devices), and the rest of the interrogator may be located at a second location, with a wire tethering the transducer or transducer array to the rest of the interrogator.

The specific design of the transducer array depends on the desired penetration depth, aperture size, and size of the individual transducers within the array. The Rayleigh distance, R, of the transducer array is computed as:

$$R = \frac{{D}^{2} - {λ}^{2}}{4 λ} \approx \frac{{D}^{2}}{4 λ} , {D}^{2} ≫ {λ}^{2}$$

wherein D is the size of the aperture and A is the wavelength of ultrasound in the propagation medium (i.e., the tissue). As understood in the art, the Rayleigh distance is the distance at which the beam radiated by the array is fully formed. That is, the pressure filed converges to a natural focus at the Rayleigh distance in order to maximize the received power. Therefore, in some embodiments, the implantable device is approximately the same distance from the transducer array as the Rayleigh distance.

The individual transducers in a transducer array can be modulated to control the Raleigh distance and the position of the beam of ultrasonic waves emitted by the transducer array through a process of beamforming or beam steering. Techniques such as linearly constrained minimum variance (LCMV) beamforming can be used to communicate a plurality of implantable devices with an external ultrasonic transceiver. See, for example, Bertrand et al., Beamforming Approaches for Untethered, Ultrasonic Neural Dust Motes for Cortical Recording: a Simulation Study, IEEE EMBC (August 2014). In some embodiments, beam steering is performed by adjusting the power or phase of the ultrasonic waves emitted by the transducers in an array.

In some embodiments, the interrogator includes one or more of instructions for beam steering ultrasonic waves using one or more transducers, instructions for determining the relative location of one or more implantable devices, instructions for monitoring the relative movement of one or more implantable devices, instructions for recording the relative movement of one or more implantable devices, and instructions for deconvoluting backscatter from a plurality of implantable devices.

Optionally, the interrogator is controlled using a separate computer system, such as a mobile device (e.g., a smartphone or a table). The computer system can wirelessly communicate to the interrogator, for example through a network connection, a radiofrequency (RF) connection, or Bluetooth. The computer system may, for example, turn on or off the interrogator or analyze information encoded in ultrasonic waves received by the interrogator.

### Communication Between an Implantable Device and an Interrogator

The implantable device and the interrogator wirelessly communicate with each other using ultrasonic waves. The implantable device receives ultrasonic waves from the interrogator through one or more ultrasonic transducers on the implantable device, and the ultrasonic waves can encode instructions for operating the implantable device. Vibrations of the ultrasonic transducer(s) on the implantable device generate a voltage across the electric terminals of the transducer, and current flows through the device, including the integrated circuit. The current can be used to charge an energy storage circuit, which can store energy to be used to emit an electrical pulse, for example after receiving a trigger signal. The trigger signal can be transmitted from the interrogator to the implantable device, signaling that an electrical pulse should be emitted. In some embodiments, the trigger signal includes information regarding the electrical pulse to be emitted, such as frequency, amplitude, pulse length, or pulse shape (e.g., alternating current, direct current, or pulse pattern). A digital circuit can decipher the trigger signal and operate the electrodes and electrical storage circuit to emit the pulse.

In some embodiments, ultrasonic backscatter is emitted from the implantable device, which can encode information relating to the implantable device, the electrical pulse emitted by the implantable device, or a detected physiological signal. For example, the ultrasonic backscatter can encode a verification signal, which verifies that electrical pulse was emitted. In some embodiments, an implantable device is configured to detect an electrophysiological signal, and information regarding the detected electrophysiological signal can be transmitted to the interrogator by the ultrasonic backscatter. To encode signals in the ultrasonic backscatter, current flowing through the ultrasonic transducer(s) of the implantable device is modulated as a function of the encoded information, such as a detected electrophysiological signal or measured physiological signal. In some embodiments, modulation of the current can be an analog signal, which may be, for example, directly modulated by the detected nerve activity. In some embodiments, modulation of the current encodes a digitized signal, which may be controlled by a digital circuit in the integrated circuit. The backscatter is received by an external ultrasonic transceiver (which may be the same or different from the external ultrasonic transceiver that transmitted the initial ultrasonic waves). The information from the electrophysiological signal can thus be encoded by changes in amplitude, frequency, or phase of the backscattered ultrasound waves.

FIG. 13 shows an interrogator in communication with an implantable device. The external ultrasonic transceiver emits ultrasonic waves (“carrier waves”), which can pass through tissue. The carrier waves cause mechanical vibrations on the miniaturized ultrasonic transducer (e.g., a miniaturized bulk piezoelectric transducer, a PUMT, or a CMUT). A voltage across the ultrasonic transducer is generated, which imparts a current flowing through an integrated circuit on the implantable device. The current flowing through to the ultrasonic transducer causes the transducer on the implantable device to emit backscatter ultrasonic waves. In some embodiments, the integrated circuit modulates the current flowing through the ultrasonic transducer to encode information, and the resulting ultrasonic backscatter waves encode the information. The backscatter waves can be detected by the interrogator, and can be analyzed to interpret information encoded in the ultrasonic backscatter.

Communication between the interrogator and the implantable device can use a pulse-echo method of transmitting and receiving ultrasonic waves. In the pulse-echo method, the interrogator transmits a series of interrogation pulses at a predetermined frequency, and then receives backscatter echoes from the implanted device. In some embodiments, the pulses are square, rectangular, triangular, sawtooth, or sinusoidal. In some embodiments, the pulses output can be two-level (GND and POS), three-level (GND, NEG, POS), 5-level, or any other multiple-level (for example, if using 24-bit DAC). In some embodiments, the pulses are continuously transmitted by the interrogator during operation. In some embodiments, when the pulses are continuously transmitted by the interrogator a portion of the transducers on the interrogator are configured to receive ultrasonic waves and a portion of the transducers on the interrogator are configured to transmit ultrasonic waves. Transducers configured to receive ultrasonic waves and transducers configured to transmit ultrasonic waves can be on the same transducer array or on different transducer arrays of the interrogator. In some embodiments, a transducer on the interrogator can be configured to alternatively transmit or receive the ultrasonic waves. For example, a transducer can cycle between transmitting one or more pulses and a pause period. The transducer is configured to transmit the ultrasonic waves when transmitting the one or more pulses, and can then switch to a receiving mode during the pause period.

In some embodiments, the backscattered ultrasound is digitized by the implantable device. For example, the implantable device can include an oscilloscope or analog-to-digital converter (ADC) and/or a memory, which can digitally encode information in current (or impedance) fluctuations. The digitized current fluctuations, which can encode information, are received by the ultrasonic transducer, which then transmits digitized acoustic waves. The digitized data can compress the analog data, for example by using singular value decomposition (SVD) and least squares-based compression. In some embodiments, the compression is performed by a correlator or pattern detection algorithm. The backscatter signal may go through a series of non-linear transformation, such as 4th order Butterworth bandpass filter rectification integration of backscatter regions to generate a reconstruction data point at a single time instance. Such transformations can be done either in hardware (i.e., hard-coded) or in software.

In some embodiments, the digitized data can include a unique identifier. The unique identifier can be useful, for example, in a system comprising a plurality of implantable devices and/or an implantable device comprising a plurality of electrode pairs. For example, the unique identifier can identify the implantable device of origin when from a plurality of implantable devices, for example when transmitting information from the implantable device (such as a verification signal). In some embodiments, an implantable device comprises a plurality of electrode pairs, which may simultaneously or alternatively emit an electrical pulse by a single implantable device. Different pairs of electrodes, for example, can be configured to emit an electrical pulse in different tissues (e.g., different nerves or different muscles) or in different regions of the same tissue. The digitized circuit can encode a unique identifier to identify and/or verify which electrode pairs emitted the electrical pulse.

In some embodiments, the digitized signal compresses the size of the analog signal. The decreased size of the digitized signal can allow for more efficient reporting of information encoded in the ultrasonic backscatter. By compressing the size of the transmitted information through digitization, potentially overlapping signals can be accurately transmitted.

In some embodiments, an interrogator communicates with a plurality of implantable devices. This can be performed, for example, using multiple-input, multiple output (MIMO) system theory. For example, communication between the interrogator and the plurality of implantable devices using time division multiplexing, spatial multiplexing, or frequency multiplexing. The interrogator can receive a combined backscatter from the plurality of the implantable devices, which can be deconvoluted, thereby extracting information from each implantable device. In some embodiments, interrogator focuses the ultrasonic waves transmitted from a transducer array to a particular implantable device through beam steering. The interrogator focuses the transmitted ultrasonic waves to a first implantable device, receives backscatter from the first implantable device, focuses transmitted ultrasonic waves to a second implantable device, and receives backscatter from the second implantable device. In some embodiments, the interrogator transmits ultrasonic waves to a plurality of implantable devices, and then receives ultrasonic waves from the plurality of implantable devices.

### EXAMPLES

The application may be better understood by reference to the following non-limiting examples, which are provided as exemplary embodiments of the application. The following examples are presented in order to more fully illustrate embodiments and should in no way be construed as limiting the scope of the application. While certain embodiments of the present application have been shown and described herein, it will be obvious that such embodiments are provided by way of example only. Numerous variations, changes, and substitutions may occur to those skilled in the art without departing from the spirit and scope of the invention. It should be understood that various alternatives to the embodiments described herein may be employed in practicing the methods described herein.

### Example 1—Greater Splanchnic Nerve Stimulation Increases Plasma Epinephrine Levels

Rats were anesthetized with isoflurane mixed with pure oxygen for the duration of the experiment. Local bupivacaine injections were given at the site of incision. Prior to making any incisions, a blood sample was taken from the submandibular vein (sample t=−30 minutes). Then, the femoral vein was cannulated in order to draw the remainder of the blood samples. A flank incision was made to access the greater splanchnic nerve close to the rib cage. The greater splanchnic nerve bundle was isolated close to the diaphragm and isolated. A bipolar nerve cuff containing 2 platinum electrodes was placed gently around the nerve bundle. Immediately prior to stimulation, a blood sample (t=0) was taken.

The greater splanchnic nerve was stimulated using biphasic (anodal-first), 1 mA constant-current pulses with a 150 μs anodal phase duration, 60 μs inter-phase interval, and 150 μs cathodal phase duration at a tonic frequency of 30 Hz for a total duration of 20 minutes in test animals (n=5). Control animals received surgery to implant the electrodes, but were unstimulated (i.e., “sham stimulation,” n=6). Additional blood samples were taken (relative to stimulation onset) at 0, 5, 20, 50, 80, 140, and 200 minutes.

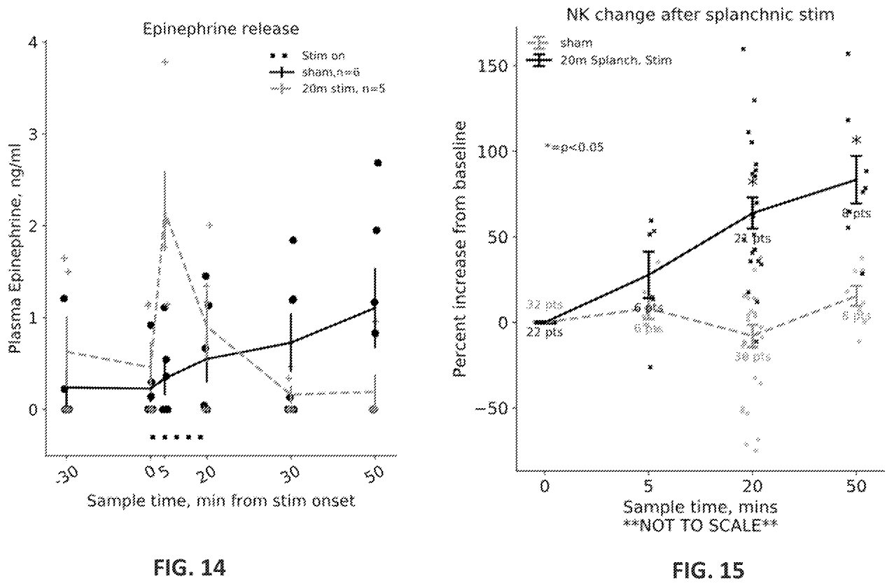

Blood samples (150 μL each) were mixed 10:1 with 200 U/mL heparin dissolved in saline. Samples were spun in a centrifuge for 20 minutes to isolate plasma, which was used to estimate plasma epinephrine concentration using ELISA. FIG. 14 shows a time course of plasma epinephrine levels in test animals (greater splanchnic stimulation, shown by cross-hairs (+)) and control animals (sham stimulation, shown by circles), which indicates that greater splanchnic nerve stimulation causes the release of epinephrine.

### Example 2—Greater Splanchnic Nerve Stimulation Increases Natural Killer Cell Circulation

Rats were anesthetized with isoflurane mixed with pure oxygen for the duration of the experiment. Local bupivacaine injections were given at the site of incision. Prior to making any incisions, a blood sample was taken from the submandibular vein (sample t=−30 minutes). Then, the femoral vein was cannulated in order to draw the remainder of the blood samples. A flank incision was made to access the greater splanchnic nerve close to the rib cage. The greater splanchnic nerve bundle was isolated close to the diaphragm and isolated. A bipolar nerve cuff containing 2 platinum electrodes was placed gently around the nerve bundle. Immediately prior to stimulation, a blood sample (t=0) was taken.

The greater splanchnic nerve was stimulated using biphasic,(anodal-first) 1 mA constant-current pulses with a 150 μs anodal phase duration, 60 μs inter-phase interval, and 150 μs cathodal phase duration at a tonic frequency of 30 Hz for a total duration of 20 minutes in test animals (n=15). Control animals received surgery to implant the electrodes, but were unstimulated (i.e., “sham stimulation,” n=16). Additional blood samples were taken (relative to stimulation onset) at 0, 5, 20, 50, 80, 140, and 200 minutes.

Blood samples (150 μL each) were mixed 10:1 with 200 U/mL heparin dissolved in saline and prepared for flow cytometry by staining the samples with natural killer (NK) cell and T cell markers. NK numbers, as percent of total lymphocytes, were determined by flow cytometric analysis. FIG. 15 shows a time course of the number of NK cells in the peripheral blood, measured as a percent of total lymphocytes for each greater splanchnic nerve stimulation test animal (shown by X) compared to sham stimulation (control, shown by circles) animals, which indicates that greater splanchnic nerve stimulation causes an increase in the number of circulating NK cells. Each individual point represents a blood sample from a single animal at the indicated time point.

### Example 3—Greater Splanchnic Nerve Stimulation for Lymphoma Treatment

Rats were anesthetized with isoflurane mixed with pure oxygen for the duration of the experiment. Local bupivacaine injections were given at the site of incision. Prior to making any incisions, a blood sample was taken from the submandibular vein (sample t=−30 minutes). A flank incision was made to access the greater splanchnic nerve close to the rib cage. The greater splanchnic nerve bundle was isolated close to the diaphragm and isolated. A bipolar nerve cuff containing 2 platinum electrodes was placed gently around the nerve bundle.

Immediately prior to stimulation, the animals received an TV (via femoral vein) bolus of fluorescently labeled YAC-1 lymphoma cancer cells (labeled with CellTrace violet, ThermoFisher), and a blood sample (t=0) was taken. The greater splanchnic nerve was stimulated using biphasic (anodal-first), 1 mA constant-current pulses with a 150 μs anodal phase duration, 60 μs inter-phase interval, and 150 μs cathodal phase duration at a tonic frequency of 30 Hz for a total duration of 20 minutes in test animals (n=7). Hemodynamic data (diastolic blood pressure, mean blood pressure, systolic blood pressure, temperature, blood oxygenation, heart rate, and blood perfusion rate) was measured during stimulation, which had a transient change at the onset of stimulation but remained within a safe range. Control animals received surgery to implant the electrodes, but were unstimulated (i.e., “sham stimulation,” n=7). An additional blood samples were taken immediately following stimulation.

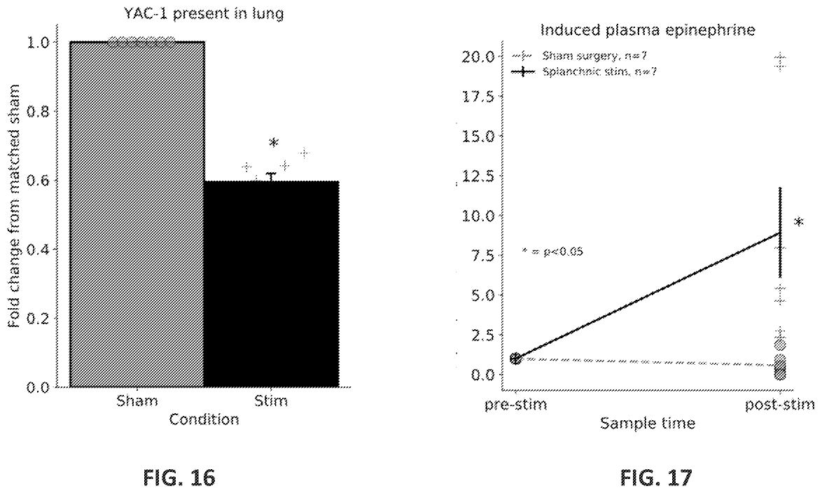

After a period of time, the animals were sacrificed and the number of YAC-1 cells present in the lung tissue of the animals was measured. Lung tissue was homogenized, red blood cells lysed, and the cells were pelleted, washed, and suspended in flow cytometry buffer. The cell suspension was run through a flow cytometer to count the total number of YAC-1 cells relative to the total number of single-cell events. The fold change in the number of YAC-1 cells detected in the lung tissue of sacrificed test (“stim”) animals is presented in FIG. 16, normalized relative to the number of YAC-1 cells detected in a matched control (“sham”) animal (control animals are self-normalized). Animals were matched to ensure surgeries and cell analysis occurred approximately at the same time, and to ensure the same YAC-1 cell culture was used. The greater splanchnic nerve stimulation resulted in a significant (p<0.05) decrease in the number of lymphoma cells detected in lung tissue.

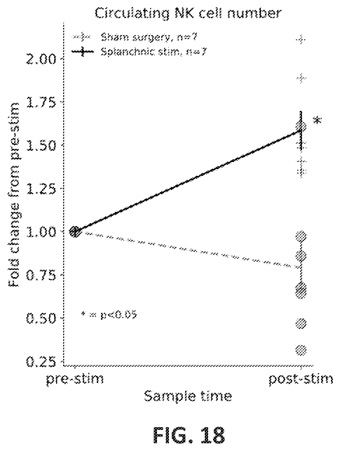

Blood samples (150 μL each) were mixed 10:1 with 200 U/mL heparin dissolved in saline. Samples were spun in a centrifuge for 20 minutes to isolate plasma, which was used to estimate plasma epinephrine concentration using ELISA. FIG. 17 shows fold-change plasma epinephrine levels in test animals (greater splanchnic stimulation, shown by cross-hairs (+)) and control animals (sham stimulation, shown by circles) before and after greater splanchnic nerve stimulation period, which indicates that stimulation causes the release of epinephrine. Additional blood samples were prepared for flow cytometry by staining the samples with natural killer (NK) cell and T cell markers. NK numbers, as percent of total lymphocytes, were determined by flow cytometric analysis. FIG. 18 shows the number of NK cells in the peripheral blood before and after the greater splanchnic nerve stimulation period, normalized to the pre-stimulation value, which indicates that greater splanchnic nerve stimulation causes an increase in the number of circulating NK cells. Splanchnic nerve stimulation is indicated by cross-hairs (+) and sham stimulation is indicated by closed circles.

### Exemplary Embodiments

The foregoing description has been described with reference to specific embodiments. Additional exemplary embodiments are provided below. However, the illustrative discussions and exemplary embodiments above are not intended to be exhaustive or to limit the disclosure to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure has been fully described with reference to the accompanying figures, it is to be noted that various changes and modifications will become apparent to those skilled in the art. Such changes and modifications are to be understood as being included within the scope of the disclosure and examples as defined by the claims.

The following embodiments are exemplary and should not be considered to limit the invention.

Embodiment 1. A method of treating a cancer in a subject, comprising electrically stimulating a thoracic splanchnic nerve of the subject with a plurality of electrical pulses emitted from one or more electrodes in electrical communication with the splanchnic nerve, wherein the plurality of electrical pulses triggers one or more action potentials in the splanchnic nerve to increase circulating natural killer (NK) cells in the subject.

Embodiment 2. A method of inhibiting cancer growth or recurrence in a subject, comprising electrically stimulating a thoracic splanchnic nerve of the subject with a plurality of electrical pulses emitted from one or more electrodes in electrical communication with the splanchnic nerve, wherein the plurality of electrical pulses triggers one or more action potentials in the splanchnic nerve to increase circulating natural killer (NK) cells in the subject.

Embodiment 3. The method of embodiment 1 or embodiment 2, wherein the subject had previously received a cancer resection surgery.

Embodiment 4. The method of any one of embodiments 1-3, wherein the thoracic splanchnic nerve is the greater splanchnic nerve, the lesser splanchnic nerve, or the least splanchnic nerve.

Embodiment 5. The method of any one of embodiments 1-3, wherein the thoracic splanchnic nerve is the greater splanchnic nerve.

Embodiment 6. The method of any one of Embodiments 1-5, wherein the electrical pulses have a current of about 100 μA to about 30 mA.

Embodiment 7. The method of embodiment 6, wherein the current is constant across the plurality of electrical pulses.

Embodiment 8. The method of any one of embodiments 1-7, wherein the electrical pulses in the plurality of electrical pulses are emitted at a frequency of about 1 Hz to about 10 kHz.

Embodiment 9. The method of any one of embodiment 1-8, wherein the plurality of electrical pulses comprises a plurality of biphasic electrical pulses.

Embodiment 10. The method of embodiment 9, wherein the biphasic electrical pulses comprise an anodal pulse phase, a cathodal pulse phase, and an inter-phase delay.

Embodiment 11. The method of embodiment 9 or 10, wherein the biphasic electrical pulses comprises an anodal phase followed by a cathodal phase.

Embodiment 12. The method of any one of embodiments 1-11, wherein the electrical pulses are about 5 μs to about 50 ms in length.

Embodiment 13. The method of any one of embodiments 1-12, wherein the plurality of electrical pulses comprises a plurality of pulse trains comprising two or more electrical pulses.

Embodiment 14. The method of embodiment 13, wherein the pulse trains are separated by a quiescent period of about 100 ms to about 15 seconds.

Embodiment 15. The method of any one of embodiments 1-14, wherein the electrical pulses in the plurality of electrical pulses are tonically emitted.

Embodiment 16. The method of any one of embodiments 1-15, wherein the splanchnic nerve is electrically stimulated by the plurality of electrical pulses for a period of about 1 minute to about 60 minutes.

Embodiment 17. The method of any one of embodiments 1-16, wherein the splanchnic nerve is electrically stimulated by the plurality of electrical pulses once daily to four times daily.

Embodiment 18. The method of any one of embodiments 1-17, wherein the one or more electrodes are operated by an implantable device fully implanted within the subject.

Embodiment 19. The method of embodiment 18, wherein the implantable device operates the one or more electrodes to emit the one or more electrical pulses based on a trigger signal.

Embodiment 20. The method of embodiment 19, wherein the trigger signal is generated by the implantable device.

Embodiment 21. The method of embodiment 19, further comprising wirelessly receiving, at the implantable device, the trigger signal.

Embodiment 22. The method of embodiment 21, wherein the trigger signal is encoded in ultrasonic waves received by the implantable device.

Embodiment 23. The method of any one of embodiments 19-22, wherein the trigger signal is based on one or more physiological signals detected within the subject.

Embodiment 24. The method of embodiment 23, wherein the implantable device comprises one or more sensors configured to detect the one or more physiological signals.

Embodiment 25. The method of embodiment 24, comprising:
receiving, at the implantable device, ultrasonic waves; and
emitting, from the implantable device, ultrasonic backscatter encoding information related to the one or more physiological signals.

Embodiment 26. The method of embodiment 25, comprising:
transmitting, from an external device, the ultrasonic waves received by the implantable device;
receiving, at the external device, the ultrasonic backscatter encoding the information related to the one or more physiological signals;
generating, at the external device, the trigger signal;
transmitting, from the external device, ultrasonic waves encoding the trigger signal; and
receiving, at the implantable device, the ultrasonic waves encoding the trigger signal.

Embodiment 27. The method of any one of embodiments 23-26, wherein the one or more physiological signals comprises an electrophysiological signal.

Embodiment 28. The method of embodiment 27, wherein the electrophysiological signal comprises an electrophysiological signal transmitted by the splanchnic nerve.

Embodiment 29. The method of any one of embodiments 23-28, wherein the one or more physiological signals comprises a temperature, a pressure, a strain, a pH, or an analyte level.

Embodiment 30. The method of any one of embodiments 23-29, wherein the one or more physiological signals comprises a hemodynamic signal.

Embodiment 31. The method of embodiment 30, wherein the hemodynamic signal comprises a diastolic blood pressure, a mean blood pressure, a systolic blood pressure, a blood oxygenation level, a heart rate, or a blood perfusion rate.

Embodiment 32. The method of any one of embodiments 1-31, comprising converting energy from ultrasonic waves received by the implantable device into electrical energy that powers the implantable device.

Embodiment 33. The method of any one of embodiments 1-32, wherein the cancer is a metastatic cancer.

Embodiment 34. The method of any one of embodiments 1-33, further comprising administering to the subject a NK cell activator.

Embodiment 35. The method of embodiment 34, wherein the NK cell activator comprises IL-2, IL-6, IL-15, or IL-12, or a bioactive fragment thereof.

Embodiment 36. The method of any one of embodiments 1-35, further comprising administering to the subject a chemotherapeutic agent.

Embodiment 37. The method of any one of embodiments 1-36, wherein the subject is a human.

Embodiment 38. A system comprising an external device and an implantable device configured to perform the method of any one of embodiments 1-37.

Embodiment 39. An implantable device comprising one or more electrodes configured to be in electrical communication with a thoracic splanchnic nerve of a subject with cancer, the device configured to operate the one or more electrodes to electrically stimulate the splanchnic nerve with a plurality of electrical pulses that triggers one or more action potentials in the splanchnic nerve that increase circulating natural killer (NK) cells in the subject.

Embodiment 40. The device of embodiment 39, comprising a substrate configured to at least partially wrap around the splanchnic nerve and position at least one of the one or more electrodes in electrical communication with the splanchnic nerve.

Embodiment 41. The device of embodiments 39 or 40, wherein the thoracic splanchnic nerve is the greater splanchnic nerve, the lesser splanchnic nerve, or the least splanchnic nerve.

Embodiment 42. The device of embodiments 39 or 40, wherein the thoracic splanchnic nerve is the greater splanchnic nerve.

Embodiment 43. The device of any one of embodiments 39-42, wherein the electrical pulses have a current of about 100 μA to about 30 mA.

Embodiment 44. The device of embodiment 43, wherein the current is constant across the plurality of electrical pulses.

Embodiment 45. The device of any one of embodiments 39-44, wherein the electrical pulses in the plurality of electrical pulses are emitted at a frequency of about 1 Hz to about 10 kHz.

Embodiment 46. The device of any one of embodiments 39-45, wherein the plurality of electrical pulses comprises a plurality of biphasic electrical pulses.

Embodiment 47. The device of embodiment 46, wherein the biphasic electrical pulses comprise an anodal pulse phase, a cathodal pulse phase, and an inter-phase delay.

Embodiment 48. The device of embodiment 46 or 47, wherein the biphasic electrical pulses comprises an anodal phase followed by a cathodal phase.

Embodiment 49. The device of any one of embodiment 39-48, wherein the electrical pulses are about 5 μs to about 5 ms in length.

Embodiment 50. The device of any one of embodiment 39-49, wherein the plurality of electrical pulses comprises a plurality of pulse trains comprising two or more electrical pulses.

Embodiment 51. The device of embodiment 50, wherein the pulse trains are separated by a quiescent period of about 100 ms to about 15 seconds.

Embodiment 52. The device of any one of embodiment 39-49, wherein the electrical pulses in the plurality of electrical pulses are tonically emitted.

Embodiment 53. The device of any one of embodiments 39-52, further comprising one or more sensors configured to detect one or more physiological signals.

Embodiment 54. The device of any one of embodiments 39-52, further comprising a body comprising a wireless communication system attached to the substrate.

Embodiment 55. The device of embodiment 54, wherein the device comprises the one or more sensors configured to detect the one or more physiological signals, and the wireless communication system is configured to wireless communicate the one or more physiological signals to a second device.

Embodiment 56. The device of embodiment 54 or 55, wherein the body is positioned on an outer surface of the substrate.

Embodiment 57. The device of any one of embodiments 54-56, wherein the wireless communication system comprises a radiofrequency (RF) antenna.

Embodiment 58. The device of any one of embodiments 54-57, wherein the wireless communication system comprises an ultrasonic transducer.

Embodiment 59. The device of embodiment 58, wherein the ultrasonic transducer is configured to receive ultrasonic waves and convert energy from the ultrasonic waves into electrical energy that powers the device.

Embodiment 60. The device of embodiment 58 or 59, wherein the device comprises the sensor configured to detect the one or more physiological signals, and wherein the ultrasonic transducer is configured to receive ultrasonic waves and emit ultrasonic backscatter encoding the one or more physiological signals.

Embodiment 61. The device of any one of embodiments 39-60, further comprising an integrated circuit configured to operate the one or more electrodes to electrically stimulate the splanchnic nerve in response to a trigger signal.

Embodiment 62. The device of embodiment 61, comprising the one or more sensors configured to detect the one or more physiological signals, wherein the integrated circuit is configured to generate the trigger signal using the one or more physiological signals.

Embodiment 63. The device of embodiment 62, comprising the wireless communication system, wherein the wireless communication system is configured to receive the trigger signal.

Embodiment 64. The device of any one of embodiments 53-63, wherein the one or more physiological signals comprises an electrophysiological signal.

Embodiment 65. The device of embodiment 64, wherein the electrophysiological signal comprises an electrophysiological signal transmitted by the splanchnic nerve.

Embodiment 66. The device of any one of embodiments 53-65, wherein the one or more physiological signals comprises a temperature, a pressure, a strain, a pH, or an analyte level.

Embodiment 67. The device of any one of embodiments 53-66, wherein the one or more physiological signals comprises a hemodynamic signal.

Embodiment 68. The device of embodiment 67, wherein the hemodynamic signal comprises a diastolic blood pressure, a mean blood pressure, a systolic blood pressure, a blood oxygenation level, a heart rate, or a blood perfusion rate.

Embodiment 69. The device of any one of embodiments 39-68, wherein the implanted device has a volume of about 5 mm³ or smaller.

Embodiment 70. A system, comprising the device of any one of embodiments 39-69 and an interrogator comprising a wireless communication system configured to wirelessly communicate with or power the device.

Embodiment 71. A pharmaceutical composition, comprising a natural killer (NK) cell activator or a chemotherapeutic agent, for use in the method of treating a cancer in a subject, or the method of inhibiting cancer growth or recurrence in a subject, according to any one of embodiments 34-37.

What is claimed is:

1. A method of treating a cancer in a subject, comprising electrically stimulating a thoracic splanchnic nerve of the subject with a plurality of electrical pulses emitted from one or more electrodes in electrical communication with the splanchnic nerve, wherein the plurality of electrical pulses triggers one or more action potentials in the splanchnic nerve to increase circulating natural killer (NK) cells in the subject.

2. A method of inhibiting cancer growth or recurrence in a subject, comprising electrically stimulating a thoracic splanchnic nerve of the subject with a plurality of electrical pulses emitted from one or more electrodes in electrical communication with the splanchnic nerve, wherein the plurality of electrical pulses triggers one or more action potentials in the splanchnic nerve to increase circulating natural killer (NK) cells in the subject.

3. The method of claim 1 or 2, wherein the subject had previously received a cancer resection surgery.

4. The method of any one of claims 1-3, wherein the thoracic splanchnic nerve is the greater splanchnic nerve, the lesser splanchnic nerve, or the least splanchnic nerve.

5. The method of any one of claims 1-3, wherein the thoracic splanchnic nerve is the greater splanchnic nerve.

6. The method of any one of claims 1-5, wherein the electrical pulses have a current of about 100 μA to about 30 mA.

7. The method of claim 6, wherein the current is constant across the plurality of electrical pulses.

8. The method of any one of claims 1-7, wherein the electrical pulses in the plurality of electrical pulses are emitted at a frequency of about 1 Hz to about 10 kHz.

9. The method of any one of claims 1-8, wherein the plurality of electrical pulses comprises a plurality of biphasic electrical pulses.

10. The method of claim 9, wherein the biphasic electrical pulses comprise an anodal pulse phase, a cathodal pulse phase, and an inter-phase delay.

11. The method of claim 9 or 10, wherein the biphasic electrical pulses comprises an anodal phase followed by a cathodal phase.

12. The method of any one of claim 1-11, wherein the electrical pulses are about 5 μs to about 50 ms in length.

13. The method of any one of claims 1-12, wherein the plurality of electrical pulses comprises a plurality of pulse trains comprising two or more electrical pulses.

14. The method of claim 13, wherein the pulse trains are separated by a quiescent period of about 100 ms to about 15 seconds.

15. The method of any one of claims 1-14, wherein the electrical pulses in the plurality of electrical pulses are tonically emitted.

16. The method of any one of claims 1-15, wherein the splanchnic nerve is electrically stimulated by the plurality of electrical pulses for a period of about 1 minute to about 60 minutes.

17. The method of any one of claims 1-16, wherein the splanchnic nerve is electrically stimulated by the plurality of electrical pulses once daily to four times daily.

18. The method of any one of claims 1-17, wherein the one or more electrodes are operated by an implantable device fully implanted within the subject.

19. The method of claim 18, wherein the implantable device operates the one or more electrodes to emit the one or more electrical pulses based on a trigger signal.

20. The method of claim 19, wherein the trigger signal is generated by the implantable device.

21. The method of claim 19, further comprising wirelessly receiving, at the implantable device, the trigger signal.

22. The method of claim 21, wherein the trigger signal is encoded in ultrasonic waves received by the implantable device.

23. The method of any one of claims 19-22, wherein the trigger signal is based on one or more physiological signals detected within the subject.

24. The method of claim 23, wherein the implantable device comprises one or more sensors configured to detect the one or more physiological signals.

25. The method of claim 24, comprising:
receiving, at the implantable device, ultrasonic waves; and
emitting, from the implantable device, ultrasonic backscatter encoding information related to the one or more physiological signals.

26. The method of claim 25, comprising:
transmitting, from an external device, the ultrasonic waves received by the implantable device;
receiving, at the external device, the ultrasonic backscatter encoding the information related to the one or more physiological signals;
generating, at the external device, the trigger signal;
transmitting, from the external device, ultrasonic waves encoding the trigger signal; and
receiving, at the implantable device, the ultrasonic waves encoding the trigger signal.

27. The method of any one of claims 23-26, wherein the one or more physiological signals comprises an electrophysiological signal.

28. The method of claim 27, wherein the electrophysiological signal comprises an electrophysiological signal transmitted by the splanchnic nerve.

29. The method of any one of claims 23-28, wherein the one or more physiological signals comprises a temperature, a pressure, a strain, a pH, or an analyte level.

30. The method of any one of claims 23-29, wherein the one or more physiological signals comprises a hemodynamic signal.

31. The method of claim 30, wherein the hemodynamic signal comprises a diastolic blood pressure, a mean blood pressure, a systolic blood pressure, a blood oxygenation level, a heart rate, or a blood perfusion rate.

32. The method of any one of claims 1-31, comprising converting energy from ultrasonic waves received by the implantable device into electrical energy that powers the implantable device.

33. The method of any one of claims 1-32, wherein the cancer is a metastatic cancer.

34. The method of any one of claims 1-33, further comprising administering to the subject a NK cell activator.

35. The method of claim 34, wherein the NK cell activator comprises IL-2, IL-6, IL-15, or IL-12, or a bioactive fragment thereof.

36. The method of any one of claims 1-35, further comprising administering to the subject a chemotherapeutic agent.

37. The method of any one of claims 1-36, wherein the subject is a human.

38. A system comprising an external device and an implantable device configured to perform the method of any one of claims 1-37.

39. An implantable device comprising one or more electrodes configured to be in electrical communication with a thoracic splanchnic nerve of a subject with cancer, the device configured to operate the one or more electrodes to electrically stimulate the splanchnic nerve with a plurality of electrical pulses that triggers one or more action potentials in the splanchnic nerve that increase circulating natural killer (NK) cells in the subject.

40. The device of claim 39, comprising a substrate configured to at least partially wrap around the splanchnic nerve and position at least one of the one or more electrodes in electrical communication with the splanchnic nerve.

41. The device of claim 39 or 40, wherein the thoracic splanchnic nerve is the greater splanchnic nerve, the lesser splanchnic nerve, or the least splanchnic nerve.

42. The device of claim 39 or 40, wherein the thoracic splanchnic nerve is the greater splanchnic nerve.

43. The device of any one of claims 39-42, wherein the electrical pulses have a current of about 100 μA to about 30 mA.

44. The device of claim 43, wherein the current is constant across the plurality of electrical pulses.

45. The device of any one of claim 39-44, wherein the electrical pulses in the plurality of electrical pulses are emitted at a frequency of about 1 Hz to about 10 kHz.

46. The device of any one of claims 39-45, wherein the plurality of electrical pulses comprises a plurality of biphasic electrical pulses.

47. The device of claim 46, wherein the biphasic electrical pulses comprise an anodal pulse phase, a cathodal pulse phase, and an inter-phase delay.

48. The device of claim 46 or 47, wherein the biphasic electrical pulses comprises an anodal phase followed by a cathodal phase.

49. The device of any one of claim 39-48, wherein the electrical pulses are about 5 μs to about 5 ms in length.

50. The device of any one of claim 39-49, wherein the plurality of electrical pulses comprises a plurality of pulse trains comprising two or more electrical pulses.

51. The device of claim 50, wherein the pulse trains are separated by a quiescent period of about 100 ms to about 15 seconds.

52. The device of any one of claim 39-49, wherein the electrical pulses in the plurality of electrical pulses are tonically emitted.

53. The device of any one of claims 39-52, further comprising one or more sensors configured to detect one or more physiological signals.

54. The device of any one of claims 39-52, further comprising a body comprising a wireless communication system attached to the substrate.

55. The device of claim 54, wherein the device comprises the one or more sensors configured to detect the one or more physiological signals, and the wireless communication system is configured to wireless communicate the one or more physiological signals to a second device.

56. The device of claim 54 or 55, wherein the body is positioned on an outer surface of the substrate.

57. The device of any one of claims 54-56, wherein the wireless communication system comprises a radiofrequency (RF) antenna.

58. The device of any one of claims 54-57, wherein the wireless communication system comprises an ultrasonic transducer.

59. The device of claim 58, wherein the ultrasonic transducer is configured to receive ultrasonic waves and convert energy from the ultrasonic waves into electrical energy that powers the device.

60. The device of claim 58 or 59, wherein the device comprises the sensor configured to detect the one or more physiological signals, and wherein the ultrasonic transducer is configured to receive ultrasonic waves and emit ultrasonic backscatter encoding the one or more physiological signals.

61. The device of any one of claims 39-60, further comprising an integrated circuit configured to operate the one or more electrodes to electrically stimulate the splanchnic nerve in response to a trigger signal.

62. The device of claim 61, comprising the one or more sensors configured to detect the one or more physiological signals, wherein the integrated circuit is configured to generate the trigger signal using the one or more physiological signals.

63. The device of claim 62, comprising the wireless communication system, wherein the wireless communication system is configured to receive the trigger signal.

64. The device of any one of claims 53-63, wherein the one or more physiological signals comprises an electrophysiological signal.

65. The device of claim 64, wherein the electrophysiological signal comprises an electrophysiological signal transmitted by the splanchnic nerve.

66. The device of any one of claims 53-65, wherein the one or more physiological signals comprises a temperature, a pressure, a strain, a pH, or an analyte level.

67. The device of any one of claims 53-66, wherein the one or more physiological signals comprises a hemodynamic signal.

68. The device of claim 67, wherein the hemodynamic signal comprises a diastolic blood pressure, a mean blood pressure, a systolic blood pressure, a blood oxygenation level, a heart rate, or a blood perfusion rate.

69. The device of any one of claims 39-68, wherein the implanted device has a volume of about 5 mm³ or smaller.

70. A system, comprising the device of any one of claims 39-69 and an interrogator comprising a wireless communication system configured to wirelessly communicate with or power the device.

71. A pharmaceutical composition, comprising a natural killer (NK) cell activator or a chemotherapeutic agent, for use in the method of treating a cancer in a subject, or the method of inhibiting cancer growth or recurrence in a subject, according to any one of claims 34-37.
